(19) United States
(12) Patent Application Publication
(10) Pub. No.: US 2018/0137943 A1
(43) Pub. Date: May 17, 2018

(54) PATIENT HANDOFF DEVICE, SYSTEM AND PREDICTIVE METHOD

(71) Applicant: MEDarchon, LLC, Nashville, TN (US)

(72) Inventors: William Baxter Webb, III, Nashville, TN (US); Nathan Charles Gonzalez, Nashville, TN (US); Ray Anthony McKelvy, Nashville, TN (US); Karol Locascio Fleming, Nashville, TN (US); Ray McKelvy, Jr., Nashville, TN (US); Caroline Kerrins Rogers, Nashville, TN (US)

(21) Appl. No.: 15/800,867

(22) Filed: Nov. 1, 2017

Related U.S. Application Data
(60) Provisional application No. 62/415,736, filed on Nov. 1, 2016.

(51) Int. Cl. G16H 80/00; G16H 40/63; G06F 21/62; G16H 50/30; G16H 10/60; G06F 17/30; H04L 29/08; G06F 3/0484; H04L 12/58

(52) U.S. Cl. CPC G16H 80/00; G16H 40/63; G06F 21/6245; G16H 50/30; H04L 51/046; G06F 17/3056; G06F 17/30283; H04L 67/148; G06F 3/04842; G16H 10/60

(57) ABSTRACT

An electronic patient handoff system and method provides a common interface for different types of healthcare providers, further customized in selection and arrangement of factors for risk mitigation. Upon initiation of patient handoff among healthcare providers, the system accesses patient data records and generates a customized patient data interface based at least on provider attributes, patient attributes and factor attributes (e.g., abnormality). The caregiver can provide feedback regarding selection and arrangement of factors in the interface, wherein the system may selectively apply the received feedback in subsequent patient handoff iterations. Potential areas of concern are listed based on provider feedback, patient-matched areas of concern, and predicted quality-adjusted life-year (QALY) impact. An intervention pathway is based on the listed potential areas of concern and represented in association with the patient data interface, with elements of the intervention pathway prioritized based on their respective predicted QALY impacts or selected patient-specific outcomes.

A portion of the disclosure of this patent document contains material that is subject to copyright protection. The copyright owner has no objection to the reproduction of the patent document or the patent disclosure, as it appears in the U.S. Patent and Trademark Office patent file or records, but otherwise reserves all copyright rights whatsoever.

### CROSS-REFERENCES TO RELATED APPLICATIONS

This application claims benefit of U.S. Provisional Patent Application No. 62/415,736, filed Nov. 1, 2016, and which is hereby incorporated by reference.

### BACKGROUND

The present invention relates generally to a system for exchanging medical information. More particularly, this invention relates to a server-based system which effectively exchanges patient-related messages based on message content, routes messages to optimal recipients based on message-forwarding algorithms, monitors and reads the status of messages after they have been sent, alters healthcare providers when a high priority message has not been read, and performs message analysis to determine if a breakdown in communication between healthcare providers is likely to occur.

Communications breakdowns are one of the greatest causes of fatal and nonfatal medical errors in the United States. An estimated 200,000 fatal medical errors occur each year, 70% of which are attributable to a breakdown in communications between healthcare providers. Many of these communications breakdown are systematic in nature; one in seven messages is sent to the wrong clinician, and thirty percent of all messages go unanswered by the recipient. Furthermore, paging can interrupt clinician workflows between ten and twenty times per hour, causing clinicians to spend more time processing communications than performing direct patient care. As such, healthcare providers often fear interrupting physicians with pages even when a problem is occurring.

As a result, messages will often go undelivered, any many that are delivered are ignored because the healthcare provider is not in a position to read or act upon the message. Therefore, critical messages pertaining to a patient's healthcare often get lost amid the din of digital communications, often resulting in fatal medical errors.

Communications breakdowns are particularly prevalent in the clinical process of patient handoff between healthcare providers. Patient handoffs are particularly critical to patient safety as up to 50% of communication breakdown occurs during this step. Problems with handoff include non-standardization of process, inaccurate information transfer, and information loss. Researchers have observed non-standardization of process in the medium used for the handoff and in the information exchanged between providers. Additionally, studies observing the length of handoff per patient have found wide variability, ranging from fractions of a second to six minutes. Importantly, electronic tools designed to aid in the handoff process have been found in clinical studies to reduce the time required to complete a handoff, variability of reporting, and adverse events that occur.

Even where there is a standard process in place, investigators have observed that 11.76% of the data that nurses are supposed to transfer during handoff is omitted. Further, 12.36% of the data transferred contains inaccuracies. Similar problems exist for physicians: while sign out sheets are commonly used for handoff between physicians, clinical studies have found that these sheets contain at least some inaccurate information 67% of the time.

If information is accurately conveyed verbally, information loss can still occur both with the individual directly receiving the handoff and in subsequent handoffs. One of the only studies to examine the extent to which the individual receiving the handoff immediately retained that information, found instant memory loss of 43.4%. Surprisingly, an intervention using a mnemonic slightly worsened retention although this was not statistically significant. Two different investigators have examined the degradation of important information through multiple cycles of handoffs when different methods of handoff were used (verbal only, verbal with note taking, electronic documentation). By the time the information reached the fifth handoff, little to no data from the initial handoff was conveyed correctly via the “verbal only” method. While note-taking can help in improving accuracy and completeness, it still pales in comparison to electronic methods.

Electronic medical records can address some of the aforementioned problems, but because of the increased amount of data and information collected about each patient, clinicians are more likely to suffer from information fatigue and fail to identify and transmit critical information. This further increases the likelihood of communications breakdowns resulting in medical error.

The Situation, Background, Assessment, and Recommendation technique (SBAR), among other known tools, can help to address communication problems. However, there are notable deficiencies with these tools. Many are onerous to complete, difficult to discern, fail to show temporal trends and do not use analytics to ensure effective handoffs are occurring.

### BRIEF SUMMARY

Exemplary embodiments of systems and methods as disclosed herein address aforementioned problems in patient handoff, providing powerful temporal representations showing patient severity, risk, important developments, and goals over time. Additionally, exemplary systems and methods as disclosed herein help track precautions and next steps, and also unifies nurse and physician input into one common interface, which bolsters intra and inter-role communication. Accordingly, the patient handoff process may be standardized while still adapting the process to fit the unique needs of each user (e.g., an ICU nurse cares about significantly different things than a surgeon).

In one aspect, a system and method according to the present disclosure pulls data from various sources (telemetry systems, laboratory systems, radiology systems, clinical documentation system, etc.) and unifies these into a single patient record.

When a clinician initiates the handoff process or clicks on the clinical summary page for a patient, a number of models may be run to determine what should be displayed to the user. For example, the model may examine user attributes (role, specialty, age, etc.), patient attributes (location, condition, adult vs. peds vs. neonate) and factor attributes (normal vs. abnormal) to determine which factors to display. Determining whether a factor is abnormal or normal may be implemented via cross-referencing with a hosted database of clinical intelligence, built on FDA data and other data gathered from the clinical literature.

In an embodiment, only the factors above a threshold value are included in the “Clinical Summary” page of the handoff interface, but the user (caregiver) can also easily navigate to see all clinical information captured through the integration API, wherein in another aspect the user can vote up and vote down information. The feedback from this exercise may then be used to update the system.

In another aspect, the method may determine if the model itself needs to be updated or if the way the user, patient or factor is categorized needs to be changed. For instance, the clinical intelligence database may fail to account for a relationship between a medication the patient is on and a laboratory value the clinician thinks is important, or it could be that data gathered from the clinical documentation system is inaccurate (up to 15% of information in the EMR is inaccurate due to being out of date or incorrect data entry) and the clinician knows the correct state and the importance of a related variable (e.g. a diagnosis or lab) to that correct state. In these cases, the model does not need to be updated but rather the information used to populate the model does. Accordingly, the method may use logistic regression to predict if the change in voting status is due to the model needing to be updated or an update needing to be made to a source of “truth.” In the case of the former, the model is updated. In the case of the latter, an alert is generated.

In addition to determining what factors to show, the two dimensional ordering of these factors may also be important. In an embodiment, the method accounts for changes based upon user role type (e.g., doctors and nurses think through and talk through patient cases differently), user specialty (surgeons move through cases differently than medical specialists), patient attributes (e.g. patients in critical condition or ICU are discussed differently from less sick patients) and even how abnormal a factor is (a really high potassium value will be discussed first especially if it was recently discovered).

In another aspect, the clinician may be enabled to provide feedback to the model regarding a preferred number of columns and ordering of data. Such feedback may be generated through navigation tools enabling the clinician to change the number of columns through a menu setting, drag and drop sections (e.g. lab results, precautions, clinical status) across the columns and order specific factors (e.g. potassium level, white blood cell count, etc. within lab results) within a section.

In a separate iteration of the display of clinical data, a system and method as disclosed herein may use a proprietary database that maps various diagnoses, labs, radiological imaging results, procedures, and tubes/lines/drains to a proprietary coding system that corresponds to different areas of the body. An embodiment of the user interface may overlay a 2D representation of the human body with this data. Accordingly, the interface can present the data which is meaningful to clinicians and helps bolster understanding. Additionally, the system and method can examine the dates of various data points to produce a video and sliding scale that the clinician can use to understand the evolution of a factor over time. This may for example be useful to nurses to know where the patient has had access points before, and to clinicians in understanding what procedures and tests have been done in the past.

In another embodiment, the presentation of factors may be modified to optimize for patient specific outcomes (e.g. medical errors, length of stay, readmissions, cost of care, patient satisfaction, an amalgamated score accounting for all of these things) rather than clinician preference.

After the clinician understands the patient's clinical background, an embodiment of a patient handoff system and method as disclosed herein may further provide an assessment. This risk mitigation activity is designed to help answer the questions: what is currently wrong with this patient, what could go wrong and what do I need to be on the lookout for?

Unfortunately, it is the part of handoff most often left out and after a long shift of caring for multiple patients it can be difficult to think critically about what needs to be done. Risk management in healthcare is an activity that is greatly influenced by the most recent patients a provider has interacted with (short-term memory). Handoff is conventionally performed between individuals of the same role (e.g. nurse to nurse or doctor to doctor); however, there is benefit in providing a common interface to engage the entire care team in this activity. In fact, one of the reasons that medical errors happen are because clinicians are afraid to share or do not feel empowered to share risks they believe to exist.

Electronic assessment with AI guided suggestions helps to overcome this but a number of challenges exist including how are potential areas of concern identified; ordered in a user interface for clinicians to consider; and how is that list modified based upon input from clinicians, geographic variation, and temporal variation.

Embodiments of a system and method as disclosed herein generate a list of potential areas of concerns which are ranked or ordered according to a determined size of an area plot, based on for example provider feedback, area of concern matches, and predicted QALY impact.

In one example, in order to generate a list of areas of concerns a vast array of clinical data must be examined, including the presence of symptoms, the diagnosis of diseases, the results of laboratory, imaging and other tests, the findings from procedures, and the activities that a patient has upcoming (including procedures, tests and events like discharge). A system and method as disclosed may address the challenge of knowing how to process all of these data points to understand the likeliness of an area of concern (e.g. a diagnosis that we are missing) by leveraging a proprietary database of areas of concern and a list of known data points associated (via logistic regression) with that area of concern. This database may also contain the percent of patients from the entire solution population with confirmation of the area of concern who manifest the data point. Both the logistic regression model to determine which data points to include in the formula as well as the percent of the population manifesting the data point may be updated over time to provide more localized and accurate AOC Match Scores.

QALY is a common accepted measure in healthcare for determining the impact on both the duration and quality of life of medical intervention. The equation for determining the QALY Impact is: (Expected Years of Life with intervention−Expected Years of Life without Intervention)*(Quality of Life with Intervention−Quality of Life without Intervention). Unfortunately, most QALYs have only been calculated around medical interventions that are expensive (drugs, procedures) which we estimate would cover less than 20% of the issues our system would the process. Additionally, the process of determining a QALY is very labor intensive and requires the patient to provide ongoing feedback about their quality of life over a long period of time. Therefore, it various embodiments of the disclosed method, it is considered impractical to use a known table of QALYs.

Rather, an exemplary system and method as disclosed herein: (1) uses linear regression to predict the likeliness of various interventions the team caring for a patient will take if the area of concern is identified; (2) uses linear regression to predict the impact that various interventions will have on the specific patients length of life; (3) uses linear regression to explain the relationship of easily obtainable variables to quality of life; (4) uses linear regression to predict the impact of various interventions on these proxy variables for quality of life; (5) calculates the product of (2) and (4) above to calculate a theoretical QALY for each intervention for the present patient; and (6) uses the results of (1) above (likeliness of various interventions being used) to weight the product determined in (5) and create a singular QALY Score.

The Area of Concern Match and QALY Impact may form a base area to help order potential areas of concern. However, the math behind this calculation is not comprehensive and subjective feedback from the care team is preferably obtained to further refine the score. The challenge with gathering this feedback is how to factor the responses (both the weight to apply and the duration over which to apply it) from various users since each user will provide a differential level of accuracy with their subjective opinion. Unfortunately, it is difficult to determine the accuracy of individual providers by looking at patient outcomes. This is because accurate prognostication of an area concern invariably leads to risk mitigation procedures which affect the natural occurrence of the outcome state. As a result, regression equations tied to patient outcomes are optimized for specificity of the clinician's gestalt and not sensitivity.

Therefore, an embodiment of a disclosed solution uses backward chaining of logic to understand when a patient had an area concern for which no intervention was provided. The solution then creates a 2×2 table and calculates Positive and Negative Predictive Values for each user from this subgroup of patients.

When a user votes up an Area of Concern, their positive predictive value (e.g., as a positive number and thus added to the sum) is entered into an equation. When a user votes down an Area of Concern, their negative predictive value (e.g., as a negative number and thus subtracted from the sum) is entered into an equation.

In the calculation of the predictive values, a 95% confidence interval may also be calculated. In one iteration of this solution, the relative width of the 95% confidence interval to a standard is used to determine the duration of time the user's vote (and applicable predictive value) is applied to the above formula.

In an embodiment, an exemplary handoff process as disclosed herein further comprises planning what to do based upon the areas of concern identified. An intervention or group of interventions (aka task bundles or pathways) may be prioritized in the user interface based upon the aforementioned QALY impact. In another example, the interventions may be prioritized using the predicted impact of the intervention on other patient-specific outcomes (medical error, length of stay, cost of care, patient satisfaction, readmission).

Once a pathway has been selected, a system and method as disclosed herein may create patient, user and resource (e.g. CT scanner) schedules via an equation that contemplates the cost and constraint associated with each entity.

### BRIEF DESCRIPTION OF THE SEVERAL VIEWS OF THE DRAWINGS

FIG. 1A is a block diagram representing an embodiment of a system according to the present invention.

FIG. 1B is a block diagram representing an embodiment of a second portion of a system according to the present invention.

FIG. 2A is a flowchart representing an embodiment of a method according to the present invention.

FIG. 2B is a flowchart representing another embodiment of a method according to the present invention.

FIG. 3 is a flowchart representing an embodiment of a login process according to the present invention.

FIG. 4 is a flowchart representing a series of steps for routing a message to an optimal recipient according to the present invention.

FIG. 5 is a flowchart representing a series of steps for monitoring the risk of communications breakdown according to the present invention.

FIG. 6 is a flowchart representing a series of steps for creating a patient digest from associated messages according to the present invention.

FIG. 7 is a flowchart representing a series of steps for creating a patient safety analysis according to the present invention.

FIG. 8 is a flowchart representing a series of steps for creating patient analytics according to the present invention.

FIG. 9 is a modified screen shot representing an exemplary graphical user interface field according to the present invention.

FIG. 10 is a modified screen shot representing an exemplary graphical user interface field according to the present invention.

FIG. 11 is a modified screen shot representing a third exemplary graphical user interface field according to the present invention.

FIG. 12 is a modified screen shot representing a fourth exemplary graphical user interface field according to the present invention.

FIG. 13 is a modified screen shot representing a fifth exemplary graphical user interface field according to the present invention.

FIG. 14 is a flowchart representing a method for performing message analysis to determine if a breakdown in communication is likely to occur according to the present invention.

FIG. 15 is a flowchart representing a series of steps for creating a patient clinical summary in accordance with one aspect of the present invention according to the present invention.

FIG. 16 is a flowchart representing a series of steps for determining patient Areas of Concern and associated treatment pathways according to the present invention

FIG. 17 is a flowchart representing a series of steps for prioritizing patient Areas of Concern in accordance with a quality-adjusted life year (QALY) impact assessment according to the present invention.

FIG. 18 is a flowchart representing a series of steps for adjusting the prioritization of Areas of Concern based upon subjective feedback from one or more user clinicians according to the present invention.

FIG. 19 is a flowchart representing a series of steps for identifying treatment pathways and creating schedules for a selected intervention for one or more Areas of Concern according to the present invention.

FIG. 20 is a flowchart representing a series of steps for determining, for a given treatment pathway, communication events associated with a high risk of causing a communication breakdown likely to cause medical error according to the present invention.

FIG. 21 is a modified screen shot representing an exemplary comparative communications event plot according to the present invention.

### DETAILED DESCRIPTION

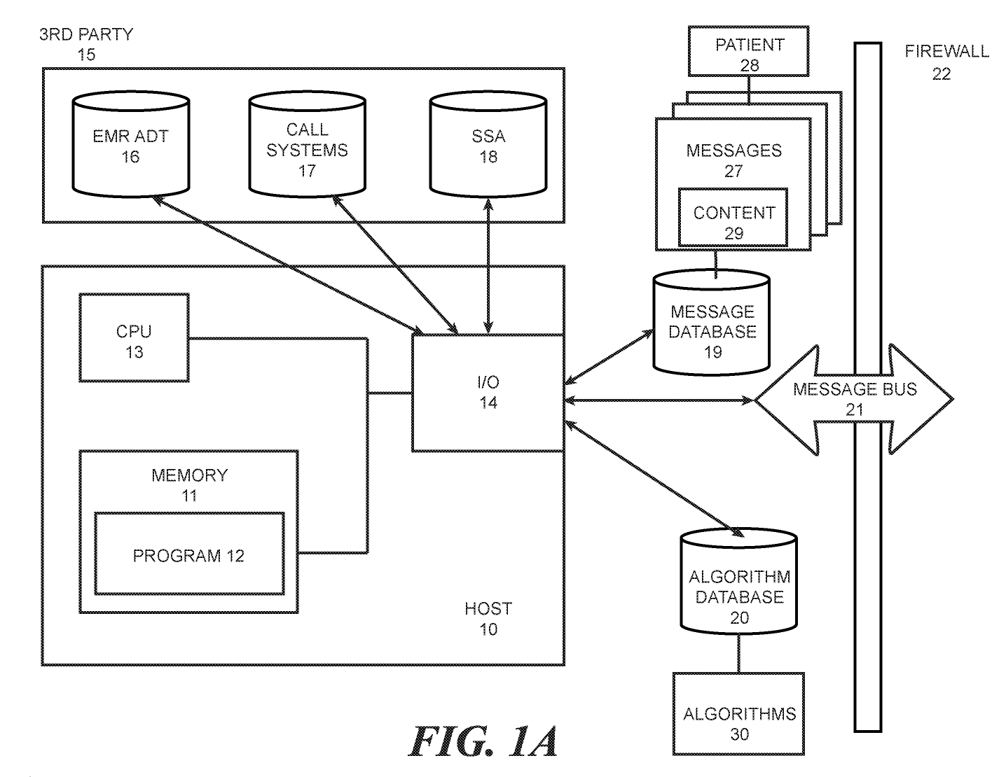

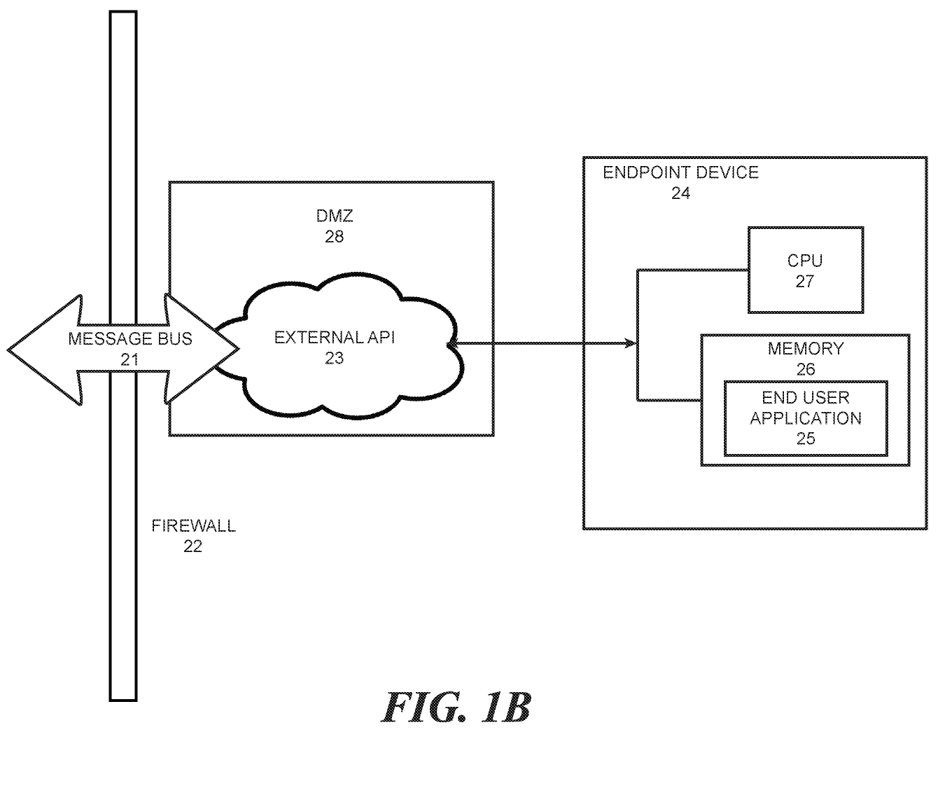

Referring generally to FIGS. 1-21, various embodiments of a system and method for exchanging medical information may be further described herein. Where the various figures may describe embodiments sharing various common elements and features with other embodiments, similar elements and features are given the same reference numerals, and redundant description thereof may be omitted below.

Throughout the specification and claims, the following terms take at least the meanings explicitly associated herein, unless the context dictates otherwise. The meanings identified below do not necessarily limit the terms, but merely provide illustrative examples for the terms. The meaning of “a,” “an,” and “the” may include plural references, and the meaning of “in” may include “in” and “on.” The phrase “in one embodiment,” as used herein does not necessarily refer to the same embodiment, although it may. Terms such as “providing,” “processing,” “supplying,” “determining,” “calculating,” or the like may refer at least to one an action of a computer system, computer program, signal processor, logic or alternative analog or digital electronic device that may be transformative of signals represented as physical quantities, whether automatically or manually initiated.

Depending on the embodiment, certain acts, events, or functions of any of the algorithms described herein can be performed in a different sequence, can be added, merged, or left out altogether (e.g. not all described acts or events are necessary for the practice of the algorithm). Moreover, in certain embodiments, acts or events can be performed concurrently, e.g. through multi-threaded processing, interrupt processing, or multiple processors or processor cores or on other parallel architectures, rather than sequentially.

The various illustrative logical blocks, modules, and algorithm steps described in connection with embodiments of a system disclosed herein can be implemented as electronic hardware, computer software, or combinations of both. To clearly illustrate the interchangeability of hardware and software, various illustrative components, blocks, modules, and steps may be described generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various illustrative logical blocks and modules described in connection with the embodiments disclosed herein can be implemented or performed by a machine, such as a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor can be a microprocessor, but in the alternative, the processor can be a controller, microcontroller, or state machine, combinations of the same, or the like. A processor can also be implemented as a combination of computing devices, e.g., a combination of a DSP and a microprocessor, a plurality of microprocessors, one or more microprocessors in conjunction with a DSP core, or any other such configuration.

The steps of a method, process, or algorithm described in connection with the embodiments disclosed herein can be embodied directly in the hardware, in a software module executed by a processor, or a combination of the two. A software module can reside in RAM memory, flash memory, ROM memory, EPROM memory, EEPROM memory, registered, hard disk, a removable disk, a CD-ROM, or any other form of computer-readable medium known in the art. An exemplary computer-readable medium can be coupled to the processor such that the processor can read information from, and write information to, the memory/storage medium. In the alternative, the medium can be integral to the processor. The processor and the medium can reside in an ASIC. The ASIC can reside in a user terminal. In the alternative, the processor and the medium can reside as discrete components in a user terminal.

The term “user interface” as used herein may unless otherwise stated include any input-output module with respect to the hosted server including but not limited to web portals, such as individual web pages or those collectively defining a host website, mobile desktop applications, telephony interfaces such as interactive voice response (IVR), and the like. Such interfaces may in a broader sense include pop-ups or links to third-party websites for the purpose of further accessing and/or integrating associated materials, data, or program functions via the hosted system and in accordance with methods of the present invention.

The term “web-based network structure” as used herein may, unless otherwise stated, refer generally to a platform effective to implement web-transitory functions, whether browser-based or otherwise. In other embodiments, the host system may within the scope of the present invention include other computer-implemented platforms and networks known to those of skill in the art which are not web-based.

The term “communications network” as used herein with respect to data communication between two or more parties or otherwise between communications network interfaces associated with two or more parties may therefore refer to any one of, or a combination of any two or more of, telecommunications networks (whether wired, wireless, cellular, or the like), a global network such as the Internet, local networks, network links, Internet Service Providers (ISPs), and intermediate communications interfaces.

The term “healthcare provider” as used herein may refer to any person or group of persons who provide medical services associated with a patient including but not limited to clinicians, physicians, dentists, radiologists, optometrists, chiropractors, pharmacists, physician assistants, nurses, dieticians, therapists, psychologists, emergency medical technicians, and paramedics.

The term “user” and/or “clinician user” as used herein may include, for example, individual healthcare providers, a group of healthcare providers, a healthcare patient/consumer/recipient/caregiver, or may refer instead to any other entity that may require access to the messaging system.

Conditional language used herein, such as, among other, “can,” “might,” “may,” “e.g.,” and the like, unless specifically stated otherwise, or otherwise understood within the context as used, is generally intended to convey that certain embodiments include, while other embodiments do not include, certain features, elements, and/or states. Thus, such conditional language is not generally intended to imply that features, elements, and/or states are in any way required for one or more embodiments or that one or more embodiments necessarily include logic for deciding, with or without author input or prompting, whether these features, elements, and/or states are included or are to be performed in any particular embodiment.

Referring first to FIG. 1, various embodiments of the host system 10 as disclosed herein include a computer-readable medium 11 having program module 12 with processor-executable instructions embodied thereon. The medium 11 may generally be effective to store data accessible to a processor 13 to which the medium 11 may be operatively linked. When the medium 11 is operatively coupled to a processor 13 the instructions may be executed by the processor 13 to perform various functions recited herein.

In an embodiment as shown in FIGS. 1A and 1B, a host system 10 includes the medium 11 operatively connected to a processor 13 effective to execute the instructions stored upon the program module 12. The medium 11 and processor 13 may be communicatively coupled to one or more input and/or output (I/O) devices 14. The I/O device 14 can include devices, for instance, but not limited to a modem for accessing another device, system, or network; a transceiver, a telephonic interface, a bridge, or a router. The I/O device 14 may be communicatively connected to one or more databases including a message database 19 effective to store one or more messages 27 associated with a patient 28, the messages comprising content 29; and an algorithm database 20 effective to store one or more algorithms 30 for processing the handling and routing of messages 27 and/or message content 29. The I/O device 14 may further be communicatively connected to third party databases 15 which may include an Admission, Discharge and Transfer System of an Emergency Medical Records database 16, one or more Call Systems databases 17, and a Single Sign-On Authentication database 18.

The host system 10 may be communicatively connected to one or more endpoint devices 24 by means of a message bus 21. The message bus can include hardware or software bus network structure connections between the host system 10 and the endpoint devices 24 and may be effective to exchange data across a firewall 22 that isolates the host system 10. The message bus may further facilitate a secure connection between the endpoint device 24 and the host system 10, the secure connection including but not limited to secure socket layer (SSL) connection. The endpoint device 24 may include a second non-transitory processor-readable memory medium (hereinafter referred to as the endpoint memory) 26 having an end user application program 25 with processor-readable instructions embodied thereon. The endpoint memory 26 may generally be effective to store data accessible to a second endpoint device processor 27 to which the endpoint memory 26 may be operatively linked. When the endpoint memory 26 is operatively coupled to the second processor 27 the instructions may be executed by the second processor 27 to receive data from the message bus 21. The instructions may be executed by the second processor 27 according to instructions provided from an external API 23 that exists outside the firewall 22 in relation to the host system 10. The external API 23 may exist on a demilitarized host 28 to which the endpoint device 24 is communicatively connected. The demilitarized host 28 may include one or more logical servers, physical computer servers, or combinations thereof. The endpoint device may be effective to provide data received from the message bus 21 and through the external API 23 to an end user application 25 stored upon the endpoint memory 26.

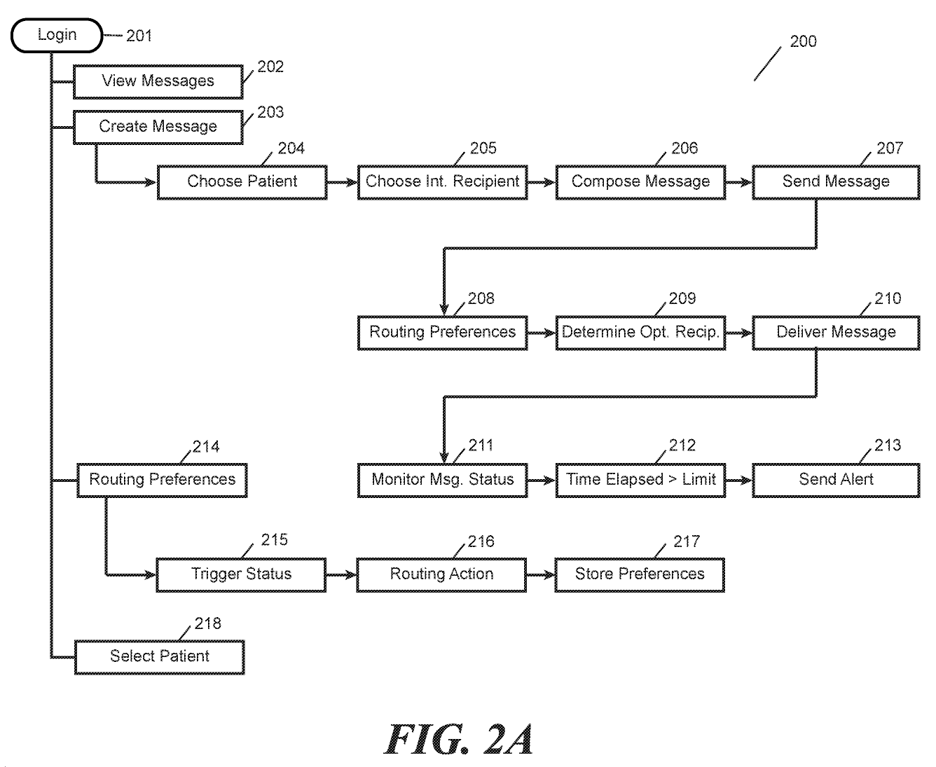

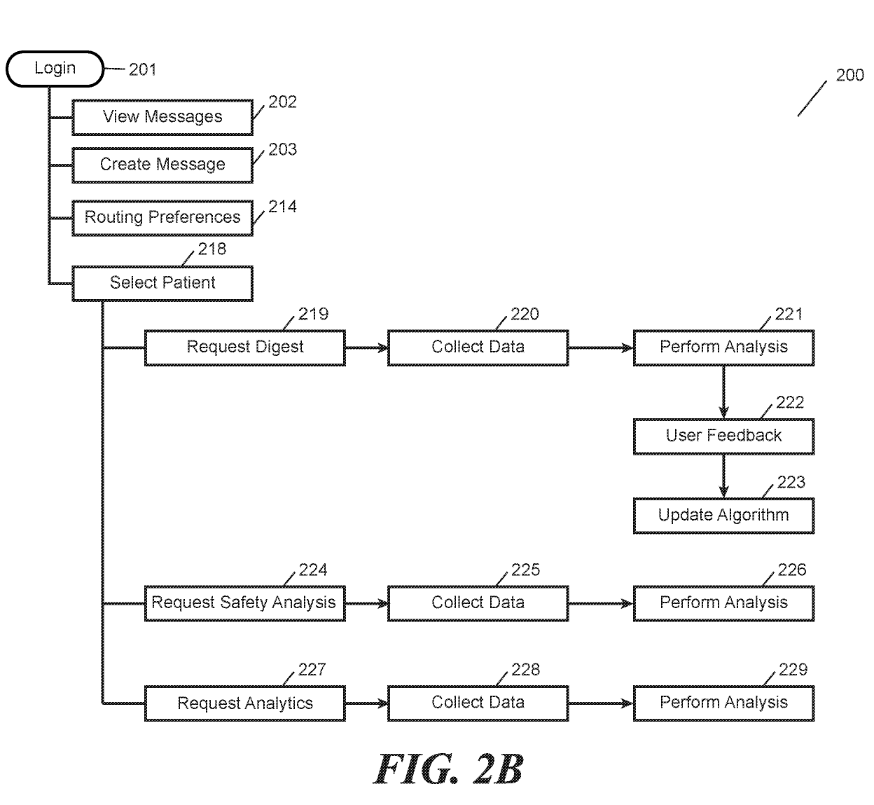

An embodiment of a messaging method 200 may be described in association with the host system represented in FIGS. 1A and 1B herein with respect to FIG. 2A. The method 200 begins at step 201 by a user successfully logging into the host system 10. In an embodiment, the user successfully logs in by establishing a secure connection between an endpoint device 24 and the host system 10, sending login credentials to the host system 10, and having the host system 10 authenticate the login credentials and grant access to the securely connected endpoint device 24.

Upon establishing a successful login, the host system 10 may present the user with several options for interacting with the messaging system. In various embodiments, the user may choose to view one or more messages associated with the user as further defined in step 202. If the user chooses to view messages, the host system 10 may provide a list of messages with which the user is associated. The messages may be associated to the user by being associated with patients with which the user is associated. In various embodiments, some or all of the associated messages may be presented along with their content through a graphical user interface as rendered by the end user application 25 of the endpoint device 24. In certain embodiments, messages may be displayed as a truncated list with a summary of message content including the name of the patient with whom the message is associated, the date and time at which the message was sent, the title subject of the message, the priority of the message, and whether or not the message has been read.

A user may choose to create a message, whereupon the method 200 proceeds to step 203 by presenting the user with a message creation function. In certain embodiments, the host system may present the message creation function through a graphical user interface as rendered by the end user application 25 of the endpoint device 24. The method 200 then proceeds to step 204 by determining a patient of the user's choice with whom to associate the new message. In various embodiments, a user may be presented with a list of associated patients. The host system 10 may determine the list of associated patients by requesting a list of patients from the electronic medical records system 16 and the message database 19. The host system 10 may combine the list of patients determined from the electronic medical records system 16 and the message database 19 into a unified, non-redundant list to display to the user. This unified, non-redundant list may be streamed across the message bus 21 to the user's endpoint device 24 in a transitory manner so that the list may not be retained upon the endpoint device following termination of the login session as described in step 201 above. In an alternative embodiment, the user may choose a patient not initially associated with the user from a list of all patients associated with the electronic medical records system 16 and message database 19.

In one embodiment, the electronic medical records system may store information regarding which users have accessed patient data through the host system 10 in accordance with the method 200 as necessary to create a HIPAA audit log. The host system 10 may therefore forward access information to the electronic medical records system 16.

Upon determination of a selected patient, the method 200 then proceeds to step 205 by determining one or more recipients for the new message. In various embodiments, the one or more recipients may be chosen from a list of healthcare providers associated with the selected patient. The host system 10 may determine the list of associated recipients by requesting a list of healthcare providers associated with the selected patient from the electronic medical records system 16, the call systems database 17, and the message database 19. The host system 10 may combine the list of healthcare providers determined from the electronic medical records system 16, the call systems database 17, and the message database 19 into a unified, non-redundant list to display to the user. This unified, non-redundant list may be streamed across the message bus 21 to the user's endpoint device 24 in a transitory manner so that the list may not be retained upon the endpoint device following termination of the login session as described in step 201 above. In an alternative embodiment, a user may choose one or more recipients not initially associated with the patient from a list of all healthcare providers associated with the host system 10 and call systems database 17.

The user may then compose the message by populating the message with message content (step 206). In various embodiments, the user may compose a message with content consisting of alphanumeric text characters. In another embodiment, the user may compose a message with content consisting of electronically stored audio, imagery, or video. In certain embodiments, the message content may include a message title subject, a message body of content, and a message priority. The message priority may be selected from a list of predetermined priority levels. This selection may be made by the user or alternatively may be automatically determined by the host system 10 from the message content according to a natural language processing algorithm stored on the algorithm database 30. In an embodiment, the message composition may occur within the end user application 25 on the user's endpoint device. The message content may be stored for purposes of composition on an external API 23 of a demilitarized host 28 so that none of the message content persists in the endpoint memory 26 following termination of the login session as described in step 201 above.

Upon completion of message composition, the method 200 then proceeds to step 207 wherein the message is sent by the user. In an embodiment, the message may be sent according to instructions sent from the user's endpoint device 24 across a message bus 21 to the host system 10. The host system 10 may write the message 27 and message content 29 to the message database 19.

The method 200 continues in step 208 by determining whether any associated routing preferences. In various embodiments, healthcare providers may have associated routing preferences that determine optimal routing procedures according to various criteria. Criteria may include date and/or time that the message has been sent, the geospatial location of the healthcare provider or the healthcare provider's endpoint device, the presence or absence of certain wireless communications networks visible to the healthcare provider's endpoint device, and the priority of the message. For example, a healthcare provider may have routing preferences defined for when the healthcare provider is not on site at the hospital to route messages to a healthcare provider on call. In another example, a healthcare provider may have routing preferences defined to receive messages of high priority during non-business hours but to route messages of low priority to an on-call physician or nurse. In an embodiment, a healthcare provider's routing preferences may be defined by the healthcare provider. In an alternative embodiment, the healthcare provider's routing preferences may be automatically determined by the host system 10 according to one or more algorithms 30 stored on the algorithms database 20.

Upon determination of the recipient's routing preferences, the method 200 proceeds to step 209 by determining the optimal recipient for the message. In various embodiments where a recipient has routing preferences defined, the host system 10 determines the optimum recipient for the message according to the selected recipient's routing preferences. The routing preferences may be stored as one or more algorithms 30 in the algorithm database 20, the call systems database 17, or both. In an embodiment wherein the recipient does not have routing preferences defined, the host system 10 may determine that the selected recipient is the optimal recipient for the message.

The method 200 continues to step 210 by sending the message to the optimal recipient as determined prior in step 209. In various embodiments, the host system 10 logically associates the message stored upon the message database 19 with the optimal recipient. The host system may send a notification to the optimal recipient regarding the existence of the message on the message database 19. In an embodiment, the message content is retained on the message server 19 and is streamed by the host system 10 across a message bus 21 to an external API 23 to which the optimal recipient's endpoint device 24 connects. The host system 10 may stream content in this manner in a transitory state so that none of the message content persists in the endpoint memory 26 following termination of the login session as described in step 201 above. Notification may occur by direct notification through the system or by push notification through a third-party system. Examples of third-party push notification systems include but are not limited to Apple Push Notification, Android Push Notification, Parse, and Urban Airship.

The method 200 then proceeds to step 211 wherein the host system 10 monitors the time elapsed since the message has been sent and the message status. In various embodiments the message status may include whether the message has been seen by the optimal recipient and whether the optimal recipient has responded to the message. The host system 10 may additionally determine the priority of the message and one or more time limits associated with the message priority for which the message can go unread or without recipient response. For example, a high priority message may have a time limit specifying that the message must be read within 2 minutes since the message was initially sent and must be responded to within 5 minutes since the message was initially sent, whereas a low priority message may have a time limit specifying that the message must be read within 24 hours since the message was initially sent and may not have a time limit specifying the time by which the recipient must respond to the message. In an embodiment, the one or more time limits are customizable.

The host system 10 compares the message status and the time elapsed since the message has been sent to the one or more time limits associated with the message priority (step 212). In various embodiments, if the time elapsed exceeds the time limit for the message status, the host system 10 will generate and send an alert (step 213). The alert may be generated according to one or more algorithms 30 stored in the algorithm database 20. The algorithms 30 may specify to generate and send an e-mail to a specific user, place an automated phone call to a specific user, or send a notification to a specific user according to the various embodiments described in step 210. Alternatively, if the time elapsed has not exceeded the time limit or if no time limit exists for the determined message status, the host system 10 may cease the monitoring activities of steps 210 and 211 for that message.

A user successfully logged in according to step 201 may choose to specify or update his or her routing preferences, wherein the method 200 proceeds to step 214. In various embodiments, the host system 10 may present routing preferences update function through a graphical user interface as rendered by the end user application 25 of the endpoint device 24. The user may choose a trigger status (step 215) from a list of pre-defined trigger statuses, the trigger status determining when the routing preference should be effective to the host system 10 upon the routing of messages as specified above in step 208. The trigger status may include criteria such as date and/or time that an incoming message has been sent, the geospatial location of the user or the user's endpoint device, the presence or absence of certain wireless communications networks visible to the user's provider's endpoint device, and the priority of an incoming message.

After a user has selected a trigger status, the method 200 continues to step 215 by prompting a user for a subsequent routing action associated with the trigger status. The user may choose a routing action from a list of pre-defined routing actions. For example, a user may choose to route messages received during a particular trigger status to a specific healthcare provider. Alternatively, a user may choose a general routing function without a predefined recipient, whereby the host system 10 will determine the specific recipient at the time that the message is sent to the user. For example, a user may choose to route messages to an attending physician on call, whereupon the host system 10 will determine at the time the message is sent to the user which specific healthcare provider is the physician on call by querying the call systems database 17.

The method 200 then proceeds to step 216 by committing and storing the updated preferences. In various embodiments, when the user specifies for the routing preference to be saved, the routing preference is translated into an algorithm to be stored upon the algorithm database 30 to be referenced by the host system 10 such as during step 208. In one embodiment, the algorithm may be sent from the endpoint device 24 through the message bus 21 to the host system 10 to be stored by the host system 10 on the algorithm database 30. In an alternative embodiment, the algorithm may be determined, constructed, and stored onto the algorithm database 20 by the host system 10 from data received by the endpoint device 24.

A user successfully logged in according to step 201 may choose to perform patient analysis functions for a particular patient, wherein the method 200 proceeds to step 218. In various embodiments, the host system 10 may present the patient analysis functions through a graphical user interface as rendered by the end user application 25 of the endpoint device 24. In various embodiments, the user may pre-select a patient for which the host system 10 will perform certain analytics. In alternative embodiments, such as may be represented for example in FIG. 2B, the user may choose the particular analytics function (steps 219, 224, 227) prior to determining the patient upon which the analytics function should be performed. In another embodiment, the host system 10 may update the call systems database 17 with the updated routing preferences.

If a user requests a message digest for a patient, the method 200 proceeds to step 219 by forwarding the digest request from the user's endpoint device 24 to the host system. The host system 10 may load from the algorithm database 20 a digest algorithm that instructs the host system 10 in performing a message digest search.

The method 200 proceeds to step 220 wherein the host system 10 collects messages 27 and message content 29 from the message database 19. In various embodiments, the host system 10 processes only messages 27 and message content 29 associated with the selected patient. The method 200 then proceeds to step 221 wherein the host system 10 performs analysis on the collected messages according to the message digest algorithm to determine the relative importance of the message. In various embodiments, the algorithm dictates to the host system 10 to process message characteristics including message priority, the time at which the message was sent, the recipients of the message, and the message content. The host system 10 applies the message algorithm to make a Boolean determination of the message importance according to the message characteristics. The host system 10 may therefore flag a message as important or not flag a message as important. In various embodiments, the host system 10 will output a summary list of messages to the user. In an embodiment, the host system 10 may stream the summary list of messages across the data bus 21 to the endpoint device 24 for transitory display via the end user application 25. The end user application 25 may display the summary list of messages as sorted chronologically, sorted by message priority, or sorted by other message characteristics identified by the host system 10. In various embodiments, the end user application 25 may display flagged messages, non-flagged messages, or a combination thereof. In circumstances of a combined display, the end user application may display flagged messages with more prominence than non-flagged messages for easier identification of important message content.

The method 200 then proceeds to step 222 by monitoring for user feedback regarding the message flag categorization. In various embodiments, a user may override the flagging of messages as described in step 221. For example, a user may choose to flag a non-flagged message, thereby identifying it as important, or may choose to de-flag a flagged message, thereby identifying it as unimportant. If the host system 10 receives user feedback in this manner, the host system 10 may analyze the digest algorithm used to perform the analysis and modify the algorithm to improve the sensitivity and specificity of future results incorporating said algorithm (step 223). In an embodiment, the host system 10 may adjust the relative weights of the specified characteristic variables to improve flagging accuracy. The host system 10 may employ a machine learning engine effective to improve the digest algorithm according to user feedback.

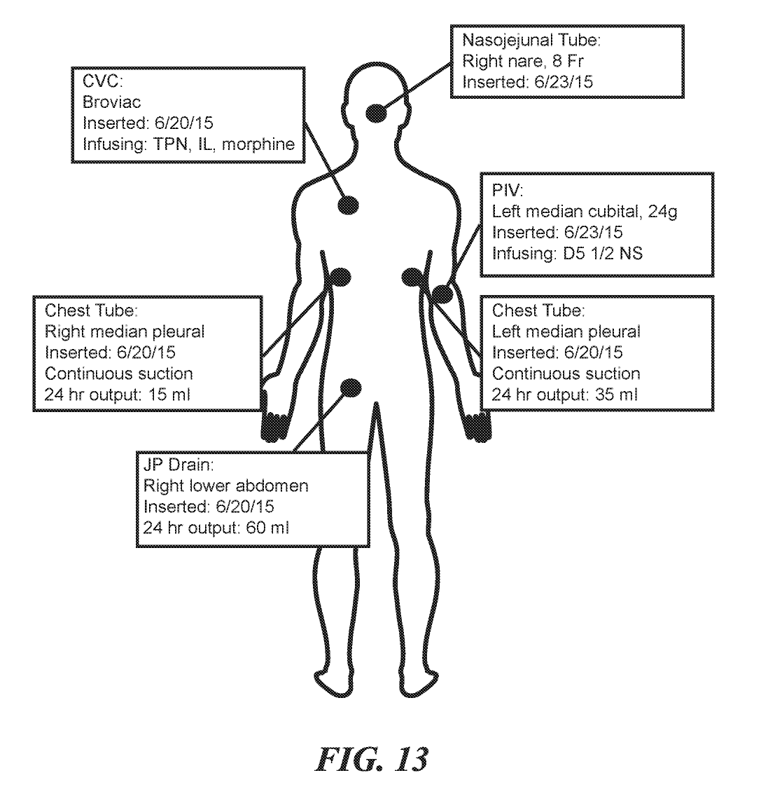

If a user requests a patient safety analysis (PSA) report for a patient, the method 200 proceeds to step 224 by forwarding the PSA report request from the user's endpoint device 24 to the host system. The host system 10 may load from the algorithm database 20 a PSA algorithm that instructs the host system 10 in performing the patient safety analysis. In an embodiment, the PSA report request and PSA algorithm (step 226) may be interpreted in accordance with FIGS. 13-18 and, namely, method 1500 as further described below.

The method 200 continues in step 225 by collecting patient data to be processed and displayed in the patient safety analysis report. In various embodiments, the host system 10 requests patient data from the electronic medical records system (16) and message data from the message database (19). The host system 10 may request patient data including patient diagnoses, patient status, treating clinicians, and care-related events associated with the patient. The host system 10 may request message data including message content, total number of messages, the time at which messages were sent, and healthcare provider participation in messages.

Upon receiving the requested data, the host system 10 analyzes the data in accordance with the PSA algorithm (step 226). Specifically, the host system 10 may calculate the likelihood of that a communications breakdown resulting in medical error will occur. The result of this analysis may be streamed to the user's endpoint device 24 for display via the end user application 25.

If a user requests patient analytics, the method 200 proceeds to step 227 by forwarding the analytics request from the user's endpoint device 24 to the host system. The host system 10 may load from the algorithm database 20 an analytics algorithm that instructs the host system 10 in performing the analytics.

The method 200 continues in step 228 wherein the host system 10 requests patient data from the electronic medical records system 16 and message data associated with the patient from the message database 19.

The method 200 proceeds to step 229 wherein the host system 10 may analyze the data in accordance with an analytics algorithm stored on the algorithm database 30. In various embodiments, the host system 10 may perform various data transformations including identifying the number of unanswered messages associated with the patient, graphing message volume over time, summarizing message volume by type over time, and comparing the volume of messages associated with the patient to a benchmark volume. The benchmark volume may be, for example, an ideal volume, an average volume, or another useful volume for comparison in determining the likelihood of a medical error occurring due to a breakdown in communication. In certain embodiments, benchmark comparisons may be made according to factors shared between other patients including the patient's first listed diagnosis, the unit in which the patient is located, the healthcare provider of the patient, and the day of hospitalization of the patient. In an additional embodiment, the data transformations may be made for more than one patient or healthcare provider so as to identify general trends and outlier data indicating a higher likelihood of medical error occurring. The data transformations may be streamed to the user's endpoint device 24 for viewing via the end user application 25.

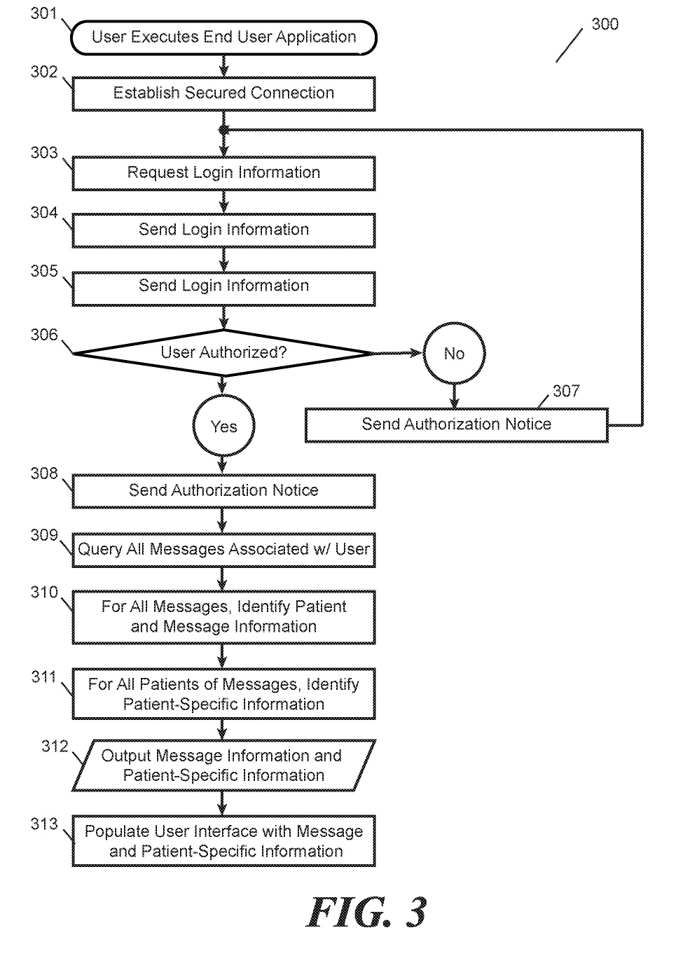

FIG. 3 demonstrates an example methodology for securely authenticating a user login from an endpoint device. The method 300 begins at a first step 301 when a user executes the end user application 25 upon the endpoint device 24. The endpoint device 24 connects to the host system 10 using a secured connection (step 302). In one embodiment the secured connection may include a secure socket layer (SSL) connection to an external API 23 residing on a DMZ host 28 communicatively coupled to the host system 10. In certain embodiments, connections may be made according to but not limited to WebSockets protocols, Server-side events protocols, or AJAX long-polling protocols. Once a secured connection has been established, the method 300 continues in step 303 by sending a request for login information over the secured connection to the endpoint device 24. In one embodiment, login information may include a user name and password associated with the user name.

The method 300 continues in step 304 by receiving the requested login information from the endpoint device 24 through the secured connection. The method 300 continues in step 305 by sending the received login information to an authentication server. In one embodiment, the authentication server may be a third-party Single Sign-On Authentication server associated with multiple healthcare login systems. In an alternative embodiment, the authentication server may be a subroutine of the host system 10.

The method 300 continues in step 306 by determining whether the endpoint device 24 is authorized to access the host system 10 by comparing the login information received to credential data associated with the authentication server. In one embodiment, successful authentication may allow the endpoint device access to additional medical systems or databases not directly associated with the host system. If authorization fails, the method 300 continues to step 307, by sending a rejection notice to the endpoint device and requests login information again according to step 303. Alternatively, if authorization is granted, the method 300 proceeds to step 308, whereupon notice of authorization is sent to the endpoint device 24.

The method 300 continues at step 309 by querying for messages associated with the user. In certain embodiments, the messages queried may represent all messages associated with a user or alternatively may represent only a subset of total messages associated with the user. In step 310, the patient associated with each message and the information associated with each message may be determined. In one embodiment, the information associated with each message may include the patient name, the message subject, the message read status, the message priority, and the message content for each message. The method 300 continues in step 311 by requesting patient-specific information for each patient identified in step 310. In one embodiment, the patient-specific information may be queried from an Emergency Medical Record system. Patient-specific information may include but is not necessarily limited to patient's demographic information, patient's medical diagnoses, patient's medical care status, clinicians treating the patient, and care-related events with which the patient is associated.

In step 312, the message information obtained step 310 and the patient information obtained step 311 is outputted to the authorized endpoint device. In one embodiment, the data is streamed in a transitory state effective to prevent any of the message information from step 310 or medical information from step 311 from persisting on an endpoint memory 26 subsequent to termination of the login pursuant to the method 300. For example, the message information and patient information may be streamed from the host system to an external API to which the endpoint device connects for the duration of the authorized login session according to the method 300, and upon termination of the method 300, the endpoint device deletes all temporary files associated with the viewing of the message information and patient information.

The method 300 continues in step 313 by populating a graphical user interface viewable by the endpoint device with the message information and patient information. In one embodiment, the graphical user interface may arrange the message information chronologically as a list of messages. In another embodiment, the graphical user interface may arrange the message information according to a list of patients with which the message information is associated. In certain embodiments, the graphical user interface may include user-selectable message lists having multiple methods of configuration, user-executable functions, and user-viewable message data.

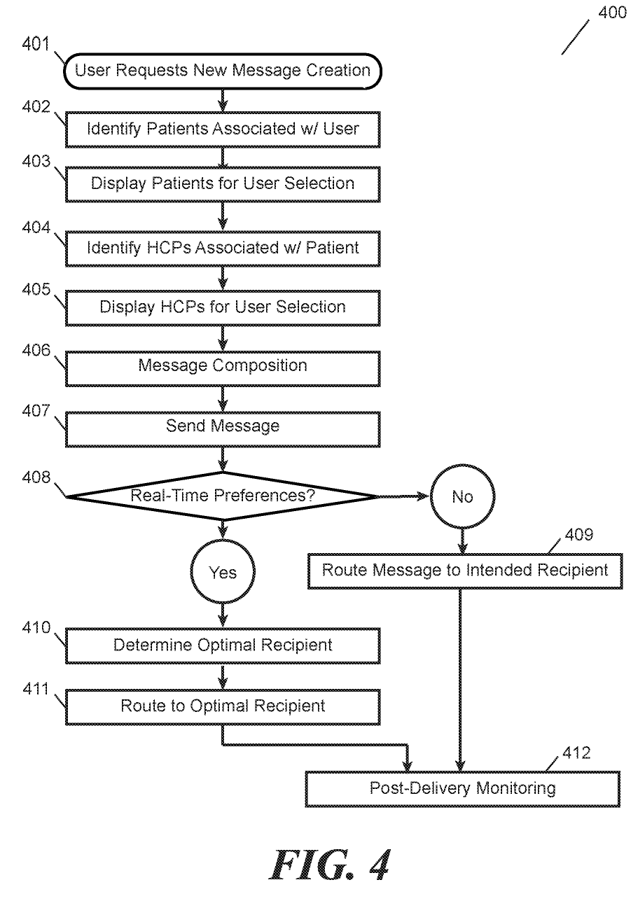

FIG. 4 is an example methodology for routing a message to an optimal recipient. The method 400 begins at step 401 when a user requests from an endpoint device 24 for a new message to be created. Upon receiving the request for a new message, the host system 10 identifies medical patients associated with a user (step 402). In certain embodiments, medical patients may be associated with a user where the user is a clinician for the patient. The method 400 continues in step 403 by displaying to the user a list of the identified patients. In certain embodiments, the user may choose one or more of the patients to whom the message will pertain. Additionally, in certain embodiments, more than one list of patients may be displayed. In an embodiment, the user may choose instead to have all patients displayed including those not determined in step 402 to be associated with the user. In another embodiment, the user may search for a specific patient wherein the host system 10 will display a list of patients associated with the user's search terms.

Once the user selects the patient or patients with whom the message is associated, the method 400 proceeds to step 405 by displaying a list of healthcare providers associated with the patient. In certain embodiments, the user may choose one or more of the healthcare providers as intended recipients of the message. Additionally, in certain embodiments, more than one list of healthcare providers may be displayed. In an embodiment, the user may choose to select a healthcare provider not among the list of healthcare providers associated with the patient. Selecting a healthcare provider in this manner may therefore associate the selected healthcare provider with the patient.

The method 400 continues in step 406 whereby the user composes a message. In one embodiment, the user may compose a text message through a graphical user interface. In alternative embodiments, the user may compose a voice message and/or a picture message. In another embodiment, the user may choose a priority level for the message. The priority level may be chosen from predetermined priorities such as “STAT,” “ASAP,” “When Possible,” or “FYI.”

The host system then determines whether the intended recipient has routing preferences (step 408). In an embodiment, an intended recipient's routing preferences are algorithms that define routing behavior for the message given certain circumstances. For example, a routing preference may specify to route the message to an alternative recipient if the recipient is within, or alternatively not within, a certain geospatial location. In certain embodiments, routing preferences may be defined according to the date, the time of day, and the geospatial location of the user and/or the user's endpoint device 24. For example, a recipient's routing preferences may declare that incoming messages should be routed to another recipient, such as the physician on call, if the user's endpoint device 24 is physically located off hospital premises as determined by GPS coordinates, or as determined by visibility of or connectivity to certain WiFi networks, or as determined by visibility or connectivity to certain cellular network towers, or as determined by a combination thereof. In another example, a recipient's routing preferences may declare that incoming messages should be routed to another recipient if the message has been sent at a specific date or time, such as at 2:00 a.m. or on weekends. In a third example, a recipient's routing preferences may declare that only messages of a certain priority should be routed to the recipient and that messages below the specified priority threshold should be routed to another recipient.

In another embodiment, the routing preferences may be defined according to whether one or more wireless communications networks are visible to the endpoint device 24 associated with the intended recipient. In additional embodiments, a recipient's routing preferences for receiving messages may be defined by the recipient. Alternatively, or in combination therewith, a recipient's routing preferences may be automatically determined through analysis of third-party systems in communication with the host system 10. For example, a recipient's routing preferences may be defined according to the recipient's hospital calendar, task list, or on-call schedule. In another example, a recipient's routing preferences may be defined according to forwarding protocols as specified by the call systems databases, such as forwarding messages to a specific individual or clinic or practice, or to an individual or clinic or practice on call for the recipient, or to a service line.

If the intended recipient does not have any routing preferences defined, then the method 400 proceeds to step 409 by routing the message to the intended recipient. In an embodiment, routing of the message associates the message with the intended recipient so that the intended recipient has visibility to the message hosted on the message database 19. In an additional embodiment, the intended recipient is notified upon receipt of the message by direct notification through the system or by push notification through a third-party system. Examples of third-party push notification systems include but are not limited to Apple Push Notification, Android Push Notification, Parse, and Urban Airship. The method then proceeds to step 412 further defined hereinafter.

Alternatively, if the intended recipient does have routing preferences defined, then the method 400 proceeds to step 410 wherein an optimal recipient is determined. In one embodiment, the optimal recipient is determined according to the routing preferences of the intended recipient. The intended recipient's routing preferences may specify to route the message to a specific individual, group, or practice. The intended recipient's routing preferences may specify to route the message to a general individual, group, or practice, whereby the host system 10 may determine which specific recipient associated with the individual, group, or practice is optimal for receiving the message. In an embodiment, routing of the message associates the message with the optimal recipient so that the optimal recipient has visibility to the message hosted on the message database 19. In certain embodiments, the message may also be associated with and/or visible to the intended recipient as well as the optimal recipient. In an additional embodiment, the optimal recipient is notified upon receipt of the message by direct notification through the system or by push notification through a third-party system. In certain embodiments, the intended recipient may also receive the same or similar notification. Examples of third-party push notification systems include but are not limited to Apple Push Notification, Android Push Notification, Parse, and Urban Airship. The method then proceeds to step 412 further defined hereinafter.

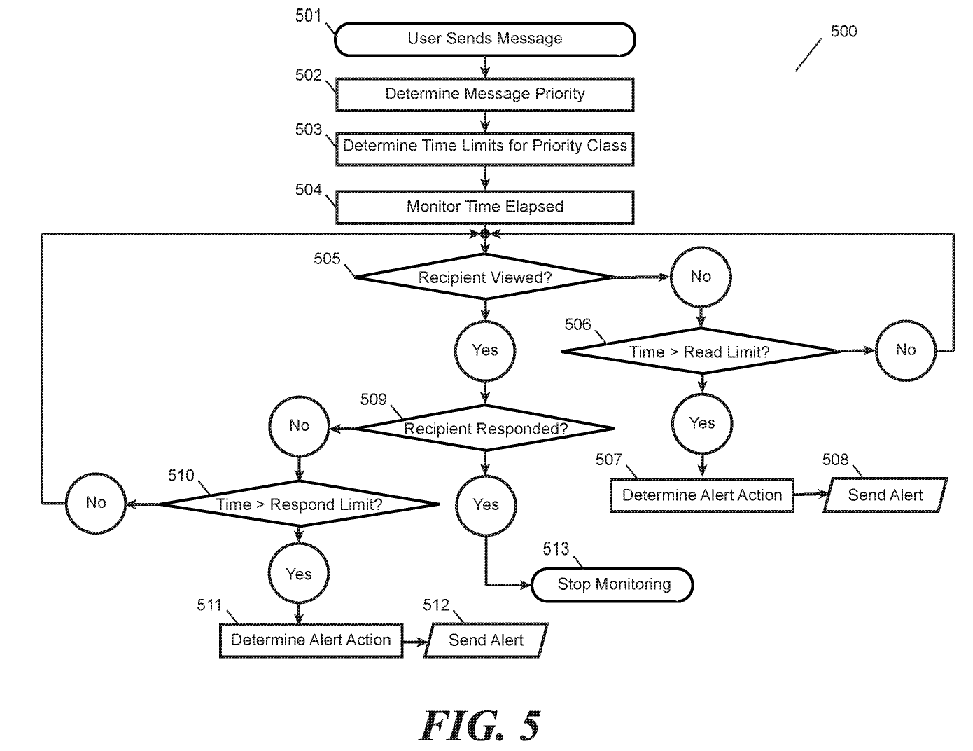

Once the message has been routed to the intended and/or optimal recipient, the method 400 proceeds to step 412 by engaging in post-delivery monitoring of the message. In various embodiments, post-delivery monitoring includes determination of whether the message has been read by the intended and/or optimal recipient and whether the intended and/or optimal recipient has responded to the message. Response to the message may include a subsequent message sent in reply or may include performance of a specific action. For example, a recipient may respond to a message by scheduling an event associated with a certain patient as determined by the host system 10. An example of a methodology for engaging in post-delivery message monitoring is further provided in FIG. 5.

FIG. 5 is an example of a methodology for monitoring the risk of communications breakdown. The method 500 begins at step 501 when a user sends a message to a recipient. In one embodiment, the message is associated with a recipient so that the recipient may view the message from an endpoint device 24. The method 500 continues in step 502 wherein the priority of the message is determined. In various embodiments, the user who composed the message may specify the priority of the message. The priority may be selected from a list of pre-defined priority statuses. For example, a user may choose a priority level for a message of “STAT,” “ASAP,” “When Possible,” or “FYI.” Alternatively, the priority of the message may be determined by the host system 10 through natural language analysis of the message content. The host system may determine the priority of a message based on the presence of certain keywords within the message content. For example, detection of the phrase “patient is coding” or “pt coding” may, according to one or more natural language processing algorithms, warrant the host system to determine a message priority of “STAT.” In an alternative embodiment, the message priority may be defined as null with no associated value.

Once the message priority has been determined, one or more time limits are determined according to the message priority level (step 503). In various embodiments, one or more priority levels may have one or more associated time limits each for the maximum time the message may go unread or for the maximum time to which a message may go without response. A higher priority level may have a shorter time limit in which a message must be read or responded to than compared to a lower priority level. A lower priority level may have a longer associated time limit or alternatively no associated time limit. For example, a message with a high priority of “STAT” may have a response time limit of 2 minutes, whereas a message with a low priority of “FYI” may have no response time limit and a read time limit of 24 hours.

The method 500 continues in step 504 wherein the host system 10 monitors the time elapsed since the message has been sent to the recipient. During this monitoring, the host system performs step 505 by determining whether or not the recipient has viewed the message. In one embodiment, the host system 10 may determine that the recipient has viewed the message where the recipient has selected, clicked on, or otherwise interacted with the message as viewed through the end user application 25 on the recipient's endpoint device 24.

If the recipient has not viewed the message, the method 500 proceeds to step 506 by comparing the time elapsed since the message was sent to the read time limit associated with the message priority level as determined in step 503 above. In one embodiment, the determined read time limit for the specified priority level may be null, wherein the host system 10 may either continue monitoring the read status of the message or may cease monitoring the read status of the message. If the time elapsed has not exceeded the read limit, then the method 500 may continue to monitor the time elapsed as further described above in step 504. Alternatively, if the time elapsed has exceeded the read limit, the host system 10 may determine an appropriate alert action according to one or more algorithms 30 stored in the algorithms database 20. In certain embodiments, the algorithm 30 may specify to send an e-mail to a user, place an automated phone call to a user, or send a notification to a user. In further embodiments, the user may be predetermined according to the algorithm 30 or alternatively determined and selected from a connected call systems database 17. In further embodiments, a notification may include direct notification through the system or by push notification through a third-party system. Examples of third-party push notification systems include but are not limited to Apple Push Notification, Android Push Notification, Parse, and Urban Airship.

Once an appropriate alert action has been determined, the method 500 proceeds to step 508 wherein an alert is sent according to the alert action determined in step 507. In one embodiment, the host system 10 may continue to monitor the message according to step 504 following sending the alert in order to determine whether to send subsequent alerts. For example, if the time elapsed exceeds a second read limit, a second alert of an escalated priority may be triggered. For example, if a recipient fails to read a message in a determined amount of time following generation of an initial alert, a subsequent alert may be sent to the recipient or to a physician on call so as to prevent the occurrence of a medical error.

If the recipient has viewed the message, the method 500 proceeds to step 509 by determining whether the recipient has responded to the message. In various embodiments, the host system may determine that the recipient has responded to the message by composing a new message associated with and in response to the first message. In another embodiment, the host system 10 may determine that the recipient has responded to the message by determining that the recipient has performed a subsequent action such as scheduling an event, filling a prescription, or admitting a patient.

If the recipient has not responded to the message, the method 500 proceeds to step 510 by comparing the time elapsed since the message was sent to the response time limit as associated with the message priority level as determined in step 503 above. In one embodiment, the determined response time limit for the specified priority level may be null, wherein the host system 10 may either continue monitoring the response status of the message or may cease monitoring the read status of the message. If the time elapsed has not exceeded the response limit, then the method 500 may continue to monitor the time elapsed as further described above in step 504. Alternatively, if the time elapsed has exceeded the response limit, the host system 10 may determine an appropriate alert action according to one or more algorithms 30 stored in the algorithms database 20. In certain embodiments, the algorithm 30 may specify to send an e-mail to a user, place an automated phone call to a user, or send a notification to a user. In further embodiments, the user may be predetermined according to the algorithm 30 or alternatively determined and selected from a connected call systems database 17. In further embodiments, a notification may include direct notification through the system or by push notification through a third-party system. Examples of third-party push notification systems include but are not limited to Apple Push Notification, Android Push Notification, Parse, and Urban Airship.

Once an appropriate alert action has been determined, the method 500 proceeds to step 511 wherein an alert is sent according to the alert action determined in step 507. In one embodiment, the host system 10 may continue to monitor the message according to step 504 following sending the alert in order to determine whether to send subsequent alerts. For example, if the time elapsed exceeds a second response limit, a second alert of an escalated priority may be triggered. For example, if a recipient fails to respond to a message in a determined amount of time following generation of an initial alert, a subsequent alert may be sent to the recipient or to a physician on call so as to prevent the occurrence of a medical error.

If the recipient has responded to the message, the method 500 may conclude at step 513 by ceasing the monitoring activities as specified above in step 504. In an alternative embodiment, the host system 10 may continue to monitor the message for additional responses for purposes of determining message analytics.

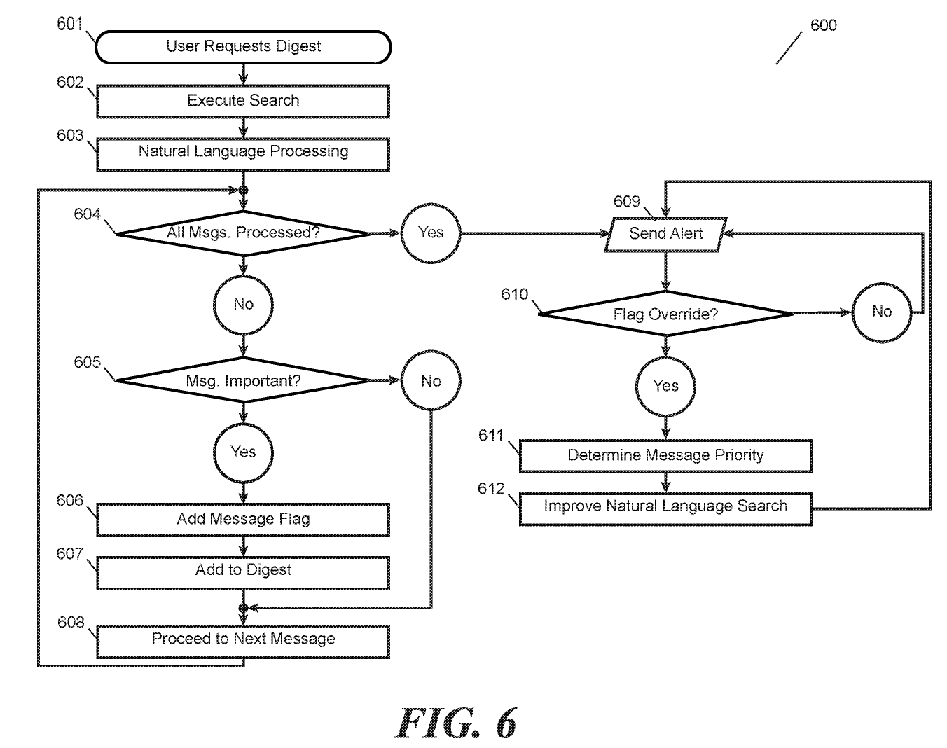

FIG. 6 is an example of a methodology for creating a digest of messages associated with a patient. The method 600 begins at step 601 when a user requests a digest and the host system 10 receives such request. In one embodiment, a user may request a digest by selecting a digest option within the end user application 25 upon the user's endpoint device 24. In an alternative embodiment, the digest may be generated automatically or periodically and without user input.

Once the host system 10 has received a digest request, the host system 10 may execute a search for all messages 27 stored on the message database 19 and associated with the patient for whom the digest was requested (step 602). In various embodiments, this search may be performed by implementing a digest algorithm from the plurality of algorithms 30 stored upon the algorithm database 20. When all associated messages have been queried, the method 600 proceeds to step 603 by applying a natural language processing algorithm to the messages 27 and associated message content 29.

The method 600 continues in step 604 by analyzing whether all of the associated messages have been analyzed with the natural language processing. If not all associated messages have been analyzed, the method continues to step 605 wherein the host system 10 determines the importance of individual messages and content of messages by processing message characteristics. Message characteristics may include the message priority, the time the message was sent, the time elapsed since the message has been sent, the recipients of the message, and the content of the message. If the host system 10 determines that the message is important, it will add a flag to the message (step 606). In various embodiments, the flag may be visible in the end user application 25 in viewing the digest summary or the message directly. The flagged message is subsequently added to the digest summary (step 607), and the host system 10 then proceeds to the next message for processing (step 608). Alternatively, if the message is not deemed important, no flag is added to the message, and the host system 10 proceeds to the next message as per step 608.

When all associated messages have been processed according to the natural language algorithm, the method 600 proceeds to step 609 wherein the digest summary is displayed, representing a collection of the most important messages associated with the patient. In various embodiments, the digest summary may be displayed as a graphical user interface upon the end user application 25 populated by the flagged message data as determined in step 606. The digest summary may be displayed as a list of flagged messages only or alternatively as a mixed list of flagged messages and non-flagged messages wherein the flagged messages have higher visual prominence so as to distinguish them to the user. In an embodiment, the digest summary may be accessible to users not associated with the patient. For example, a nurse, doctor, or health care provider on rotation not otherwise directly associated with a certain patient may be able to request and view a digest for that patient under his or her immediate care.

The method 600 continues in step 610 by determining whether a flag override has occurred. In various embodiments, the initial determination of a message flag according to step 606 may be overridden by the user. For example, a user may determine that message flagged as important should not be flagged as important, and may remove the flag from the message, or alternatively may determine that an non-flagged message deemed unimportant is important and should be flagged. In certain embodiments, a user may override the initial determination of a message flag according to step 606 by selecting or deselecting the message flag within the message list interface or digest summary interface of the end user application.

If a user overrides a message flag in this manner, the method 600 proceeds to step 611 wherein the host system 10 toggles the message flag. In various embodiments, the toggle determination is Boolean, wherein a user may turn a flag for a message off or on. The method 600 then proceeds to step 612, wherein the host system 10 analyzes the natural language processing algorithm to determine how the natural language processing algorithm's sensitivity and specificity may be improved to ensure greater accuracy with future determinations of message importance. In one embodiment, the host system 10 may improve the natural language processing algorithm automatically through application of a machine learning engine or by adjusting the relative weight of the variables used to make the determination of message importance.

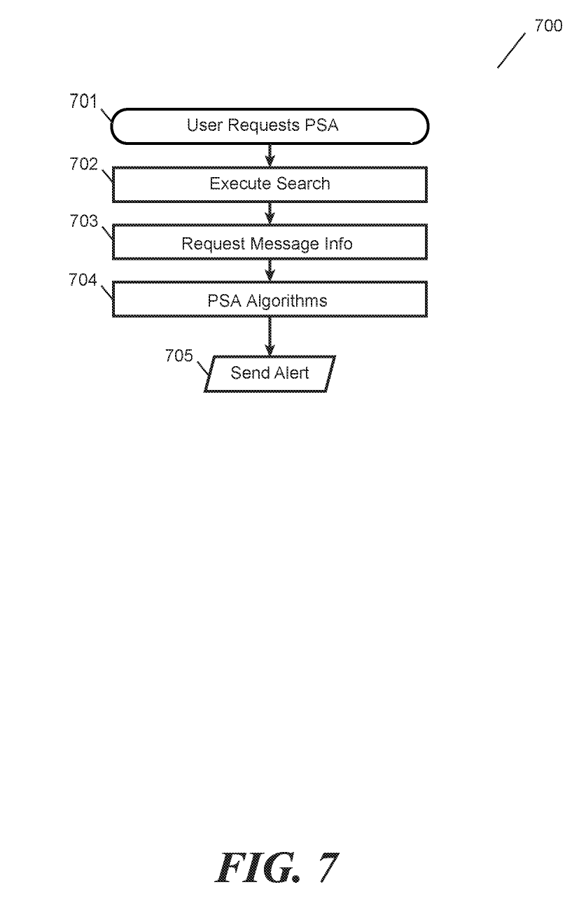

FIG. 7 is an example of a methodology for creating a patient safety analysis report associated with a patient. The method 700 begins at step 701 when a user requests a patient safety analysis (PSA) report and the host system 10 receives such request. In one embodiment, a user may request a PSA report by selecting a PSA report option within the end user application 25 upon the user's endpoint device 24. In an alternative embodiment, the PSA report may be generated automatically or periodically and without user input.

Once the host system 10 has received a PSA report request, the method 700 proceeds to step 702 by requesting patient information. In various embodiments, patient information may be requested from third-party databases 15 such as the electronic medical records database 16. Patient information may include patient demographics, patient diagnoses, patient status, clinicians treating the patient, and care-related events associated with the patient such as hospitalizations, medical emergencies, transfers, medical procedures, and releases. Patient information may additionally include insurance status, total medications, number of procedures planned, and ability or inability to communicate due to mitigating circumstances such as medication or disease. In an embodiment, additional information may be collected pertaining to the patient's health care providers such as providers' years of experience, caseload, types of cases within caseload, and hours worked.

The method 700 proceeds to step 703 by requesting message information pertaining to the patient. In various embodiments, message information may be requested from the message database 19 wherein the messages 27 are associated with the patient 28. Message information may include message content, number of messages, priority of messages, the time at which the messages were sent, and clinician participation in messages. In an embodiment, additional information may be collected pertaining to the healthcare providers' messaging such as number of communications per day, number of patient handoffs, and types of handoffs regarding source and destination department.

The host system 10 may then apply a PSA algorithm to the collected patient information and message information (step 704). The PSA algorithm may predict a likelihood that a breakdown in communication resulting in a medical error will occur. In one embodiment, the PSA algorithm may be a logistic regression formula derived from data associated with the patient and the patient's health care providers. In such an embodiment, the logistic regression formula may be functionally related to a Boolean determination of prior medical error having occurred from a communication breakdown. In another embodiment, the weight of analyzed variables may be changed or altered according to the location of the patient, location of the healthcare facility, or location of the system deployment. In another embodiment, the PSA algorithm may be a combination of one or more algorithms and steps and described in method 1400.

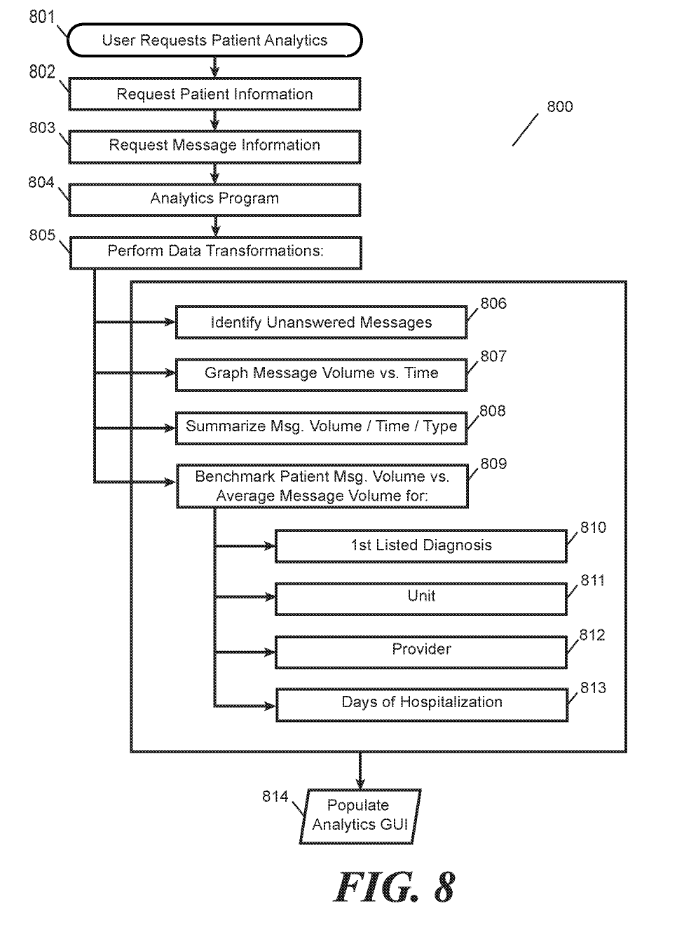

FIG. 8 is an example of a methodology for creating patient analytics report. The method 800 begins at step 801 when a user requests a patient analytics report and the host system 10 receives such request. In one embodiment, a user may request an analytics report by selecting an analytics report option within the end user application 25 upon the user's endpoint device 24. In an alternative embodiment, the analytics report may be generated automatically or periodically and without user input.

Once the host system 10 receives a request for a patient analytics report, the method continues at step 802 by requesting patient information. Patient information may include patient demographics, patient diagnoses, patient status, clinicians treating the patient, and care-related events associated with the patient such as hospitalizations, medical emergencies, transfers, medical procedures, and releases. Patient information may additionally include insurance status, total medications, number of procedures planned, and ability or inability to communicate due to mitigating circumstances such as medication or disease.

The method 800 proceeds to step 803 by requesting message information pertaining to the patient. In various embodiments, message information may be requested from the message database 19 wherein the messages 27 are associated with the patient 28. Message information may include message content, number of messages, priority of messages, the time at which the messages were sent, and clinician participation in messages.

The host system 10 may then apply an analytics algorithm to the collected patient information and message information (step 804). The analytics algorithm may then perform various data transformations to provide analytics upon the collected data. In various embodiments, the analytics algorithm may perform the steps of identifying what and how many of the messages associated with the patient are currently unanswered (step 806), graphing the volume of messages associated with the patient over a function of time (step 807), summarizing the volume of messages associated with the patient over a function of time based on message priority (step 808), and providing benchmarks regarding the number of messages associated with the patient against other patients in the hospital system (step 809). These steps may be performed individually or in any combination. In an additional embodiment, the host system 10 may further perform regressions of step 809 by providing messaging benchmarks based on patients who share the patient's first listed diagnosis (step 810), by providing messaging benchmarks based on patients within the same medical unit location as the analyzed patient (step 811), by providing messaging benchmarks based on patients who share the same health care provider as the analyzed patient (step 812), and by providing messaging benchmarks based on patient who have been hospitalized for the same number of days as the analyzed patient (step 813). In one embodiment, the benchmarks of steps 809 through 813 may provide a percentage of analyzed patient's message volume versus the average message volume of the control group over a specific period of time. In an additional embodiment, steps 809 through 813 may provide messaging benchmarks for a selected health care provider.

The method 800 then proceeds to step 814 by populating an analytics GUI with the transformed data. In one embodiment, the analytics GUI is displayed upon the end user application 25 of the user's endpoint device 24, the transformed data being streamed to the endpoint device in a non-persistent, transitory form. For example, the data may not persist on the endpoint device 24 after the session with the host system 10 is terminated. The transformed data may appear as one or more graphics color-coded to indicate threat metrics associated with a likelihood of communication breakdown resulting in medical error. In another embodiment, the analytics GUI may display transformed data for multiple patients within the same interface so as to allow the user to identify relative abnormalities that may represent a likelihood of communication breakdown resulting in medical error.

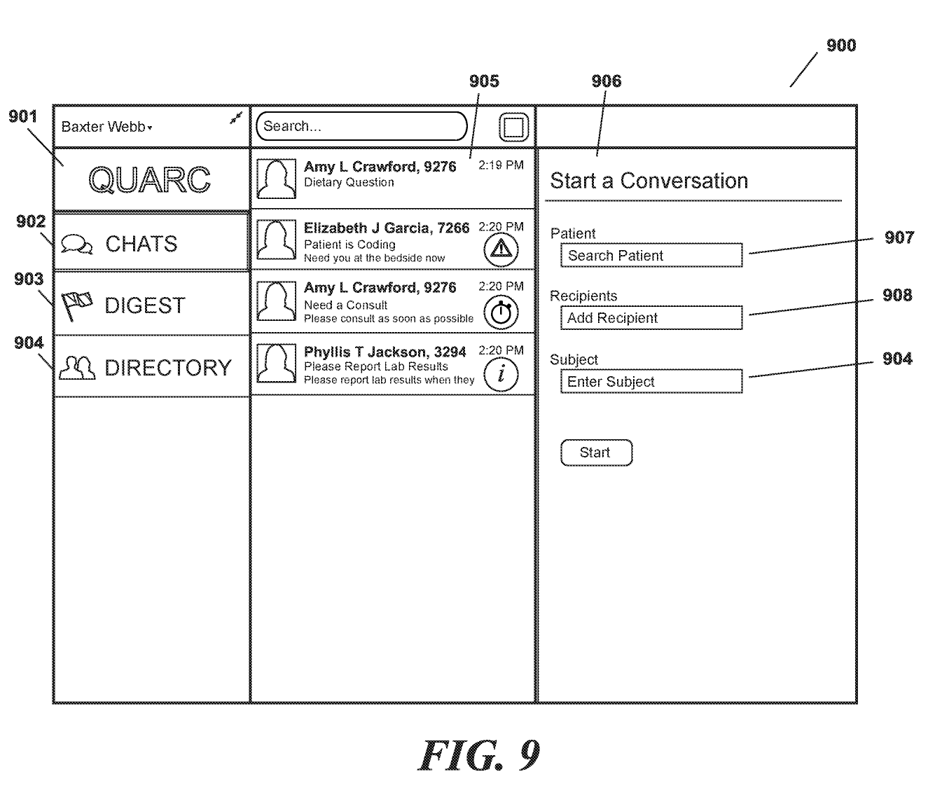

Referring now to FIGS. 1-8, in various embodiments the end user application 25 of the endpoint device 24 may generate a graphical user interface for the displaying of messages, message content, and related media for interacting with the host system 10. An example of such an embodiment may be described further with reference to FIGS. 9-13.

FIG. 9 represents a default screen 900 whereby the user can perform various messaging functions. In this embodiment, a main menu 901 presents three selectable options whereby a user may choose to open a messaging interface 902, to request a patient digest 903, or open a directory of users and/or patients 904. A second portion of the screen is populated with a list of recently received messages 905. In this configuration, the messaging interface option 902 has been selected, resulting in a third portion of the screen being populated with a new message form 906. The user may choose a patient with whom the message will be associated 907, one or more recipients for the message 908, and a title subject for the message 909.

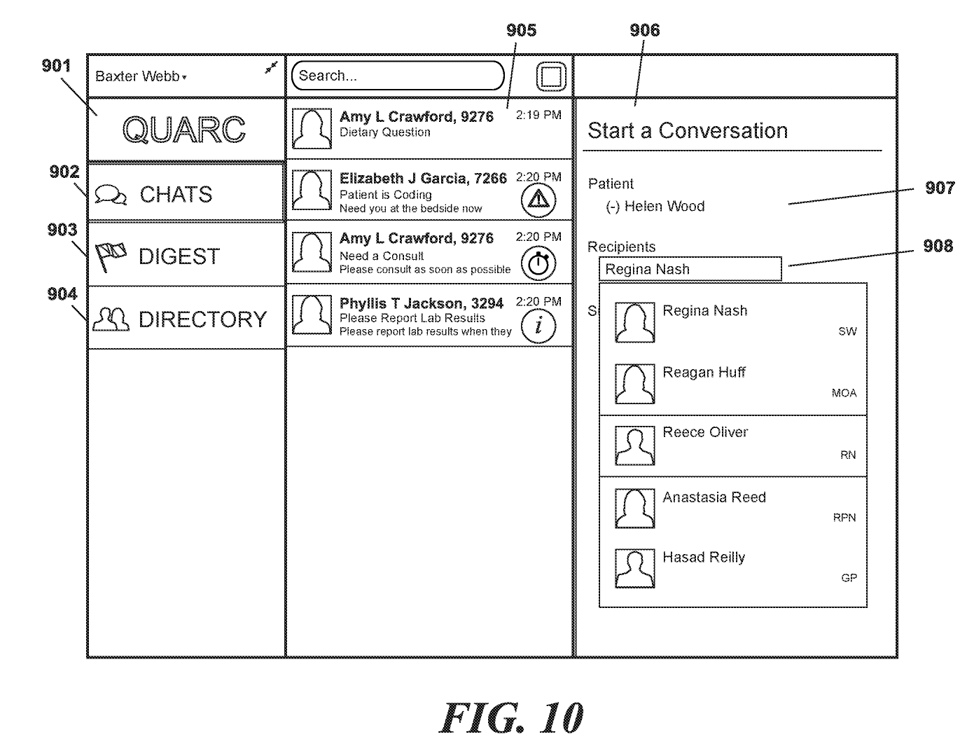

FIG. 10 represents a subsequent screen wherein the user has selected a patient 907 and may now select one or more recipients from an expanded list of recipients 908. In an embodiment, the expanded list of recipients 908 may preferably be populated only with healthcare providers associated with the selected patient 907.

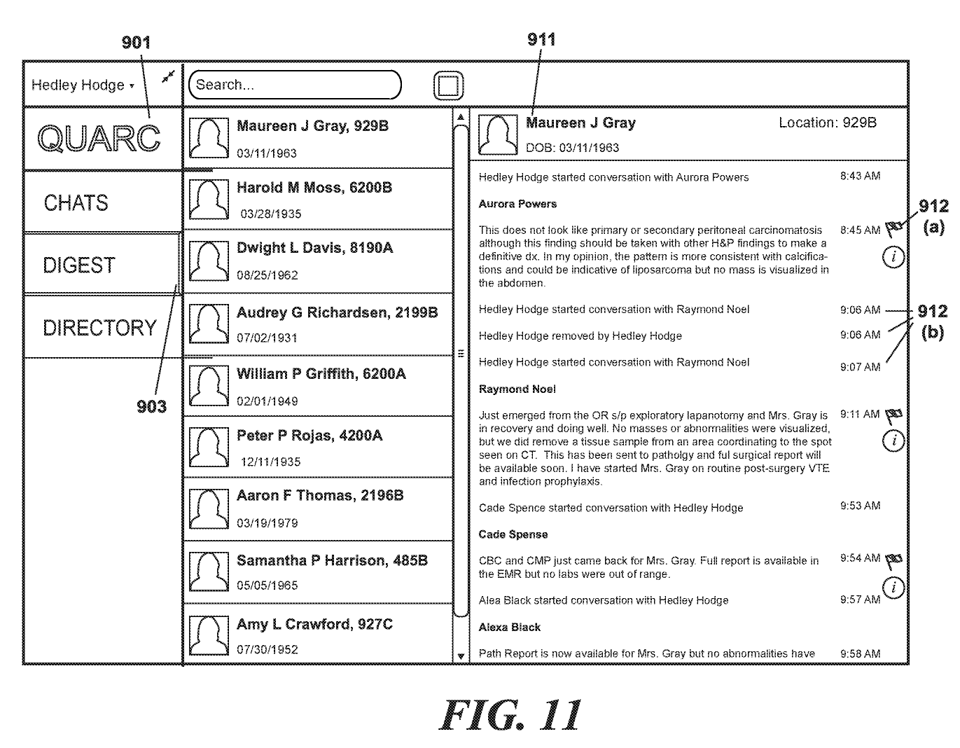

FIG. 11 represents a subsequent screenshot wherein the user has requested a patient digest 903 from the main menu 901. A second portion of the screen is populated with a list of patients 910 associated with the user. In this configuration, a patient has been selected, resulting in a third portion of the screen being populated with a digest summary 911 of messages associated with the patient. In this embodiment, the summary of messages is displayed as a chronological list wherein messages are flagged as important 912(a) or are not flagged as important 912(b).

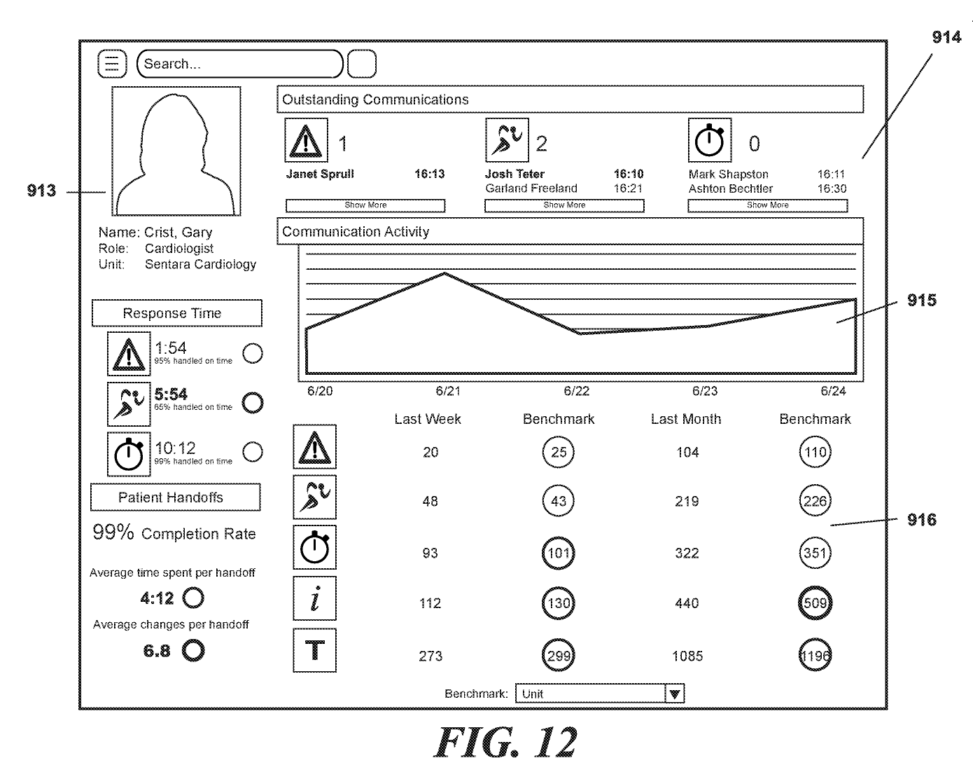

FIG. 12 represents a screenshot of a patient analytics report. In this embodiment, the name of the patient is displayed along with biographic and demographic information 913. Analytics pertaining to average response times for various message priorities is displayed with an average time of response, a percentage of timely responses, and an icon of a color corresponding to the percentage of timely responses. Additional information effective to indicate a likelihood of communication breakdown resulting in medical error may be displayed. In this configuration, additional information includes the number of messages unread or to which have not been responded separated into categories by priority 914, a graph of the volume of messages pertaining to the patient over a period of time 915, and the volume of message pertaining to the patient compared to an ideal benchmark as categorized by benchmark type, time period analyzed, and message priority 916.

FIG. 13 represents a screenshot of a patient clinical data summary as mapped to a two-dimensional representation of a human body. In this embodiment, patient-specific factors such as diagnoses; labs; radiological imaging results; procedures; and tubes, lines, and drains are mapped in accordance to associated areas of the body for the patient and displayed in accordance with their relative area. For example, a nasojejunal tube may be coded in association with the central face area and may be displayed in relative area to the central face area of the two-dimensional representation. Multiple data points may be displayed in association with the figurative representation to provide clinicians comprehensive understanding of procedures performed, patient access points, and other medical modalities.

In one embodiment, the system may cross-reference the data points with their respective dates to produce a video and sliding scale of data points over time for a patient and demonstrate the evolution of one or more factors over a period of time. For example, a clinician user may be able to scroll through a timeline and view the entry of tubes, lines, and drains for a patient across the patient's ER stay.

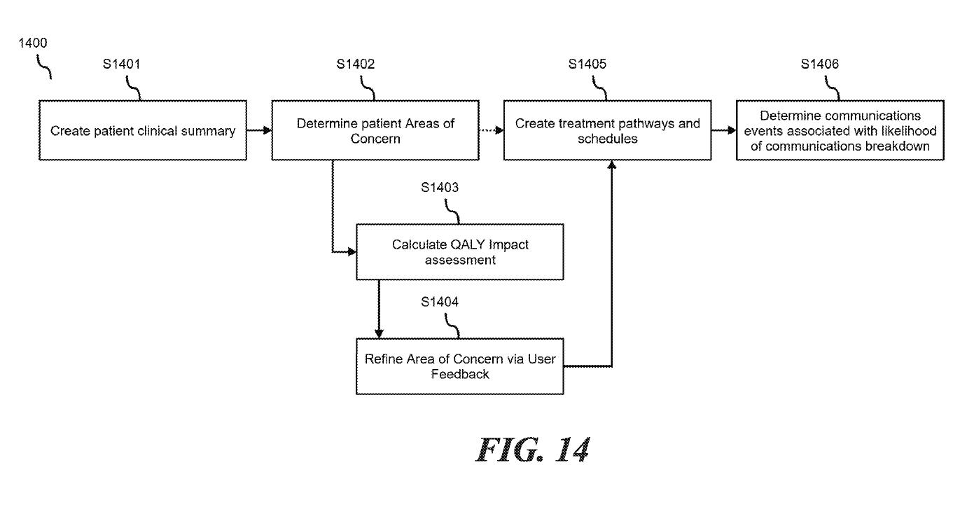

FIG. 14 is an example of a methodology for determining a likelihood of communication breakdown in accordance with one aspect of the present invention. In various embodiments, method 1400 may be interpreted in accordance with method 700 in the generation of a PSA report. In other embodiments, method 1400 may further describe a methodology for user handoff. The method begins at a first step S1401 wherein a patient clinical summary is created, such as by request of a user clinician or automatically in accordance with determination of a patient handoff between healthcare providers. In various embodiments, step S1401 may be further described in accordance with method 1500 below.

In step S1402, the system determines one or more Areas of Concern for a patient based upon regression of patient data to known Area of Concern models. In various embodiments, step S1402 may be further described in accordance with method 1600 below.

In step S1403, the system may refine the determination, display, or categorization of one or more identified Areas of Concern by calculating a QALY Impact assessment for one or more preventions associated with the identified Area of Concern. In various embodiments, step S1403 may be further described in accordance with method 1700 below.

In step S1404, the system may further refine the determination, display, or categorization of one or more Areas of Concern in accordance with clinician user feedback. In various embodiments, step S1404 may be further described in accordance with method 1800 below.

In step S1405, the system creates one or more treatment pathways and schedules for one or more of the identified Areas of Concern for a patient on the basis of particularized information for the specific patient, including factoring of costs, constraints, and availability of determinable options and resources within treatment pathways. In various embodiments, step S1405 may further be described in accordance with method 1900 below.

In step S1406, the system determines, for the one or more Areas of Concern and associated treatment pathways, predicted communications events associated with a comparatively high risk of communications breakdown and determines a likelihood of communication breakdown therefrom. In one embodiment, the system creates a predicted frequency of communications events over time for a particular patient and compares this communications plot against one or more communications plots for comparative patient populations, determines variances between the plots, analyzes the type of messages associated with the variances, and determines whether said variance is a correlated predictor and likely identifier of a communications breakdown likely to cause medical error in association with the patient. In various embodiments, step S1406 may further be described in accordance with method 2000 below.

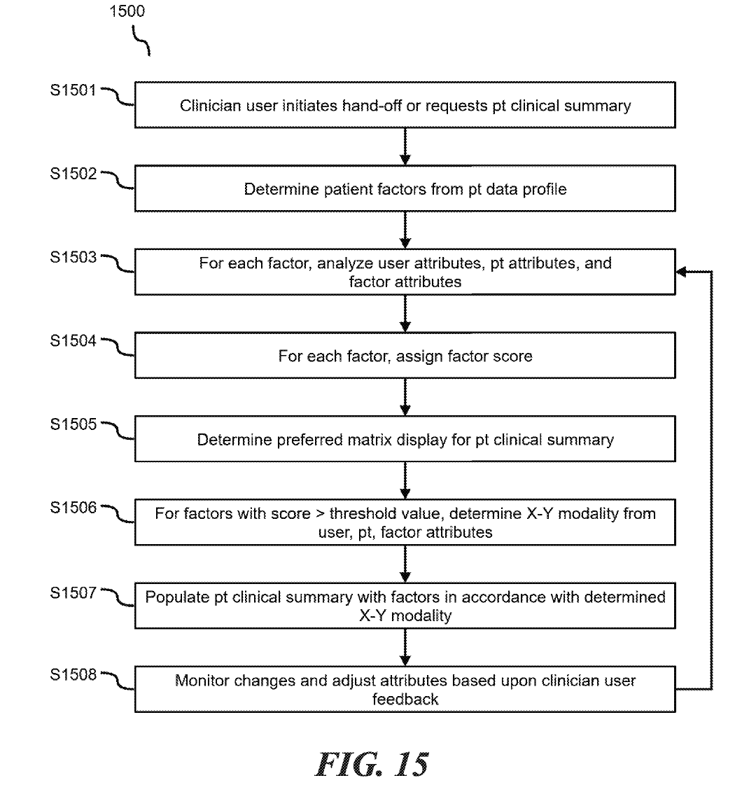

FIG. 15 is an example of a methodology for creating a patient clinical summary in accordance with one aspect of the present invention. In various embodiments, method 1500 and the patient clinical summary may be interpreted in accordance with steps 224 and 226 of method 200 and/or method 700 and may be the same or an alternative embodiment of the PSA described therein.

The method 1500 begins at a first step S1501 wherein a clinician user initiates a patient handoff, wherein the patient is transferred from the care of one healthcare provider and/or clinician to another healthcare provider and/or clinician, or requests a patient summary via the user interface. In step S1502, for the selected patient, the system determines one or more patient factors from the patient's associated data profile. In one embodiment, the patient's associated data profile may be a patient record with various patient data amalgamated from various medical systems, such as, for example, the EMR system, into a singular, periodically or continuously updated profile. In another embodiment, the system may query the various medical systems for patient information upon each iteration of method 1400. Factors may comprise, for example, medical issues, diagnoses, modalities, and other areas of medical concern.

In step S1503, the system analyzes for each patient factor associated clinician user attributes, patient attributes, and factor attributes. Clinician user attributes may include, for example, clinician profile information such as the user's role, specialty, age, and the like; and may further include clinician profile settings such as display preferences. Patient attributes may include, for example, the patient's location, condition, age, height, and the like. Factor attributes may include a binary determination of normality versus abnormality by cross-referencing the patient factor with a clinical intelligence database classifying modes of normality for patient factors. Factor attributes may further be determined in accordance with patient attributes. For example, a patient factor for blood pressure with a characteristic of 70 systolic pressure over 45 diastolic pressure may be determined as normal factor attribute for a neonate patient and abnormal factor attribute for an adolescent patient.

In step S1504, the system assigns a factor score for each factor in accordance with the factor's associated user, patient, and factor attributes. The algorithms for determination of factor score may be weighted in accordance with the attributes to maximize the score for critical factors most important to the user clinician. For example, patient factors pertaining to a patient's respiratory system may be weighted to score higher for clinician users with a user attribute indicating the clinician user is a pulmonologist. Other factors may score higher or lower in accordance with other aspects of user, patient, and factor attributes. For example, abnormal non-respiratory conditions may score higher than normal respiratory conditions for a pulmonologist user based upon severity, criticality, or degree of abnormality. The weighted scoring of patient factors may generally trend in accordance with clinician concern, such that patient factors requiring immediate attention may score higher than other factors comparatively.

In step S1505, the system determines a preferred matrix display for patient clinical summary. In one embodiment, the matrix display may be determined in accordance with clinician user attributes such as specialty, patient attributes such as criticality, and/or degree of abnormality of the patient factor. For example, one type of display model may be preferred for surgeons and another type of display model may be preferred for medical specialists; one type of display model may be preferred for ICU patients and another type of display model may be preferred for general admission. Further, highly abnormal factors may warrant the determination of a display that calls attention to the abnormal factor. Determination of display models may further be determined in accordance with a combination of one or more of user attributes, patient attributes, and factor attributes, and may further be determined in accordance with individual user settings defining display preferences. For example, user preferences may override or refine the determination of a system-preferred matrix display where the user has selected that critical information appear in a particular place upon the summary. For further example, users may pin certain types of factors for display, such as preference for always displaying a patient's heart rate in the top right pane regardless of abnormality.

In step S1506, the system determines an X-Y modality for display of factors with a score greater than a threshold value in accordance with the determined matrix display for the patient clinical summary. For example, where the system may have determined a patient clinical summary template display in a three-column, two-row grid configuration, and there are nine factors with a score above the threshold value (factors A-I in order of magnitude), the system may determine that the first row should be populated with factors A, B, and F, and that the second row should be populated with factors C, H, and G in accordance with display preferences. In said example, factors A, B, and C may be the highest scoring factors and are assigned to the top and leftmost cells in accordance with general display preferences; however, the user clinician may have set preferences to always show factor F in the top right cell and factor G in the bottom-right cell, and never to show factors D or E unless they are abnormal.

In step S1507, the system populates the clinical summary display with the patient factor information in accordance with the modality determined in S1406. Displays may in one embodiment be a two-dimensional grid of information. In other embodiments, displays may be modular graphical user interfaces rendering display in non-tabular format.

In step S1508, the system monitors for clinician user feedback and adjusts user, patient, or factor attributes in accordance with the clinician's input. For example, the clinician user may elect to see all patient factors as opposed to those provided on the patient clinical summary and may further select information to display upon the patient clinical summary, whereby the system may adjust one or more of the stated attributes to ensure current and future display of that factor on the summary. In another example, a clinician user may specify that a factor deemed normal is abnormal.

In such or similar embodiments where a user clinician provides input changing the display, the system may further determine if the change requires an update to the modeling algorithms or if the change indicates uncertainty into the veracity of the source information defining the attribute. Such change may be determined via logistic regression. For example, a clinician specifying that recent immunization records should be displayed on the summary may result in a determination that the model should be updated based upon user preference, and the clinician attributes and/or display preferences may be updated to display immunization records for patients of this type. Comparatively, if the clinician specifies that a patient's listed potassium level is abnormal when currently listed as normal, the system may determine compared to the relatively high degree of confidence in that potassium level's normality that the change in status is a question of veracity and may generate an alert requesting that the data be updated and/or verified. Clinician users may further be able to specify the format of the preferred matrix display, such as adding or removing columns and rows via the user interface.

In another embodiment, the presentation of patient factors are modified to optimize for improving patient-specific outcomes rather than clinician preference. For example, factors may be scored higher and, therefore, displayed to indicate factors related to outcomes such as, e.g., medical errors, length of stay, readmissions, cost of care, and patient satisfaction.

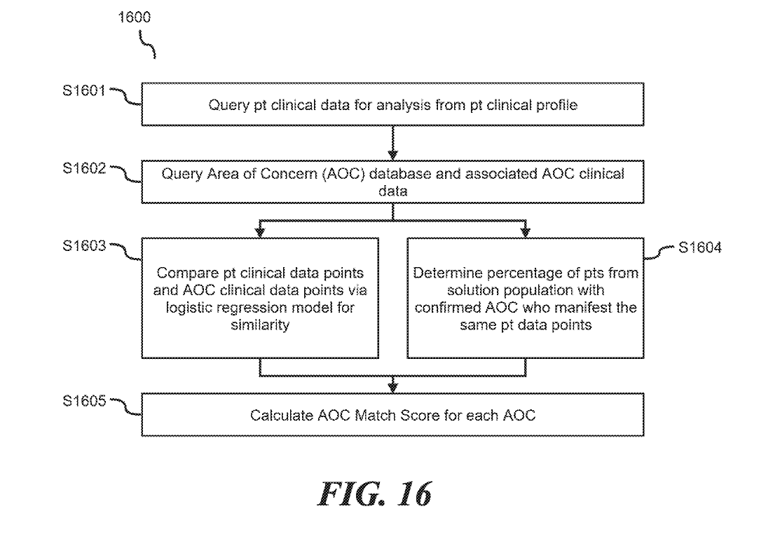

FIG. 16 is an example of a methodology for determining patient Areas of Concern and associated treatment pathways in accordance with one aspect of the present invention. In an embodiment, method 1600 may be performed in accordance with method 1400 and following the initiation of a patient handoff in method 1500.

Method 1600 begins at step S1601, wherein the system queries patient clinical data for analysis from a patient clinical profile. The patient clinical profile may be a singular profile associated with the patient and stored in a profile database or may be an amalgamated and/or consolidated data set for the patient queried from one or more medical systems, e.g. one or more EMR systems. Patient clinical data may include, for example, presence of symptoms, diagnoses of diseases, results of laboratory tests, results of imaging tests, findings from clinical procedures, upcoming patient activities, and the like.

In step S1602, the system queries an Area of Concern database containing clinical areas of concern (e.g. an undiagnosed condition) and associated clinical data for the Areas of Concern stored therein or in association therewith. Clinical data thereof may be data typical of the Area of Concern, such as, for example, typical patient symptoms and diagnostic results.

In step S1603, the system compares the patient's clinical data points with the Area of Concern database via a logistic regression to determine similarity, thereby associating with the patient high-correlation Areas of Concern. Further, the system in step S1604 determines the percentage of patients from a solution population confirmed with each respective Area of Concern who manifest the same data point or data points. Combining the results of steps S1603 and S1604, the system refines a match confidence from patient-centric logistic regression and population-centric percentage manifestation.

In step S1605, the system calculates an Area of Concern Match Score (AOC Match Score) for each Area of Concern identified and weighed in accordance with steps S1603 and S1604. For example, the system may determine based on high logistic regression AUC value two Areas of Concern, such as 0.793 and 0.814, and may further determine that the comparative percentage for each Area of Concern is 91% and 63% respectively, thereby determining the product of each as AOC Match Scores of 72.16 and 51.28 respectively. In one embodiment, the system may only calculate AOC Match Scores for Areas of Concern with logistic regression or percentage correlations above a certain confidence level. In an alternative embodiment, the system may calculate AOC Match Scores for all Areas of Concern in the Area of Concern database.

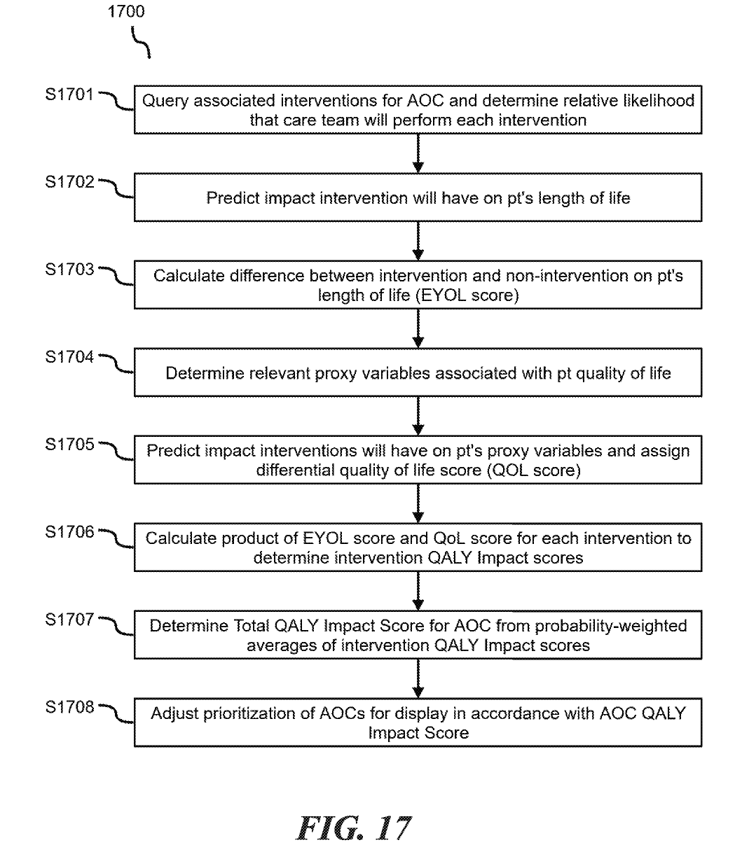

FIG. 17 is an example of a methodology for prioritizing Areas of Concern in accordance with a quality-adjusted life year (QALY) impact assessment. In various embodiments, including in accordance with method 1400, method 1700 may be performed subsequent to method 1600 for identifying Areas of Concerns to refine further the determination of patient-specific Areas of Concern. The method 1700 begins at step S1701 wherein the system queries associated interventions for one or more particular Areas of Concern and determines a relative likelihood that a care team will perform the associated intervention. In an embodiment, the determination may be made in accordance with historical patient population and clinician data wherein historical data pertaining to the performance of each intervention comparative to other interventions for the same Area of Concern. For example, the system may determine from a historical patient population diagnosed with pneumonia that one intervention pathway, prescription of antibiotics, has a 74% likelihood of occurring, and another intervention pathway, intubation, has a 12% likelihood of occurring. Determination of relative likelihoods may further be defined in accordance with population determinations. For example, the system may determine that intubation has a higher, 24% likelihood of occurring for the present patient who is over the age of 80. The determination may be made in accordance with linear regression models for each pathway for a given Area of Concern.

In step S1702, the system predicts the impact that each intervention will have on the associated patient's length of life. For example, the system may identify for a given Area of Concern three statistically significant interventions with a 70%, 20%, and 10% chance of likelihood of being performed.

In step S1703, the system calculates the expected difference between intervention and non-intervention on the patient's length of life (expected-years-of-life score, or EYOL score). In one embodiment, EYOL scores may be calculated for each intervention associated with a particular Area of Concern. Continuing the above example, the system may determine for the identified Area of Concern individual pathway EYOL scores of 7, 3.5, and 0.5 for interventions A, B, and C, respectively, wherein each intervention is predicted to extend the patient's life by 7 years, 3.5 years, and 6 months.

In step S1704, the system further determines relevant proxy variables associated with the patient's quality of life. Proxy variables may include, for example, ratings for pain, mobility, weight, activity, cognitive function, and the like. In various embodiments, proxy variables may be weighted differently for each patient in accordance with patient attributes. For example, mobility and activity ratings may be weighted higher for athlete patients than they would for non-athlete patients. In an embodiment, proxy variables may be determined in accordance with linear regression models.

Upon determination of the relevant proxy variables, in step S1705, the system predicts the impact the various interventions will have on the patient's proxy variables and assign for each intervention a differential quality of life score (QOL score). In an embodiment, prediction of impact may be determined in accordance with linear regression models. For example, the system may determine that intervention A has a total QOL score of 12.3, intervention B has a total QOL score of 8.7, and intervention C has a total QOL score of 24.8. QOL scores may in certain embodiments be patient-specific based upon proxy variable weights, such that the interventions associated with the same Area of Concern may result in different QOL score totals for another patient. For example, a factor measuring likelihood of sterility may be minimized for an octogenarian but maximized for a young adult, such that a pathway likely to increase risk of sterility would be scored much higher for the octogenarian than for the young adult.

In step S1706, the system calculates the product of the EYOL score and QOL score for each intervention to determine QALY Impact scores for each intervention. Continuing the above example, intervention A would have a QALY Impact score of 86.1, intervention B would have a QALY Impact score of 30.45, and intervention C would have a QALY Impact score of 12.4.

In step S1707, the system determines a Total QALY Impact Score for the Area of Concern weighted by the likelihood of various interventions being used. Continuing the present example, the Total QALY Impact Score for the identified Area of Concern would be 60.27+6.09+1.24=67.6.

In step S1708, the system adjusts the prioritization of Areas of Concern for display in association with the patient via the user interface, such as highlighting of Areas of Concern in a Patient Safety Analysis report, a hand-off report, or other such report or display for a patient, in accordance with the Total QALY Impact Score. Accordingly, Areas of Concern with higher likelihoods of positive outcomes may be prioritized to promote clinician understanding and selection of more effective interventions. In various embodiments, interventions may be prioritized in accordance with likelihood of performance and/or QALY Impact scores, further promoting performance of effective and beneficial interventions over less effective and less beneficial interventions for one or more given Areas of Concern.

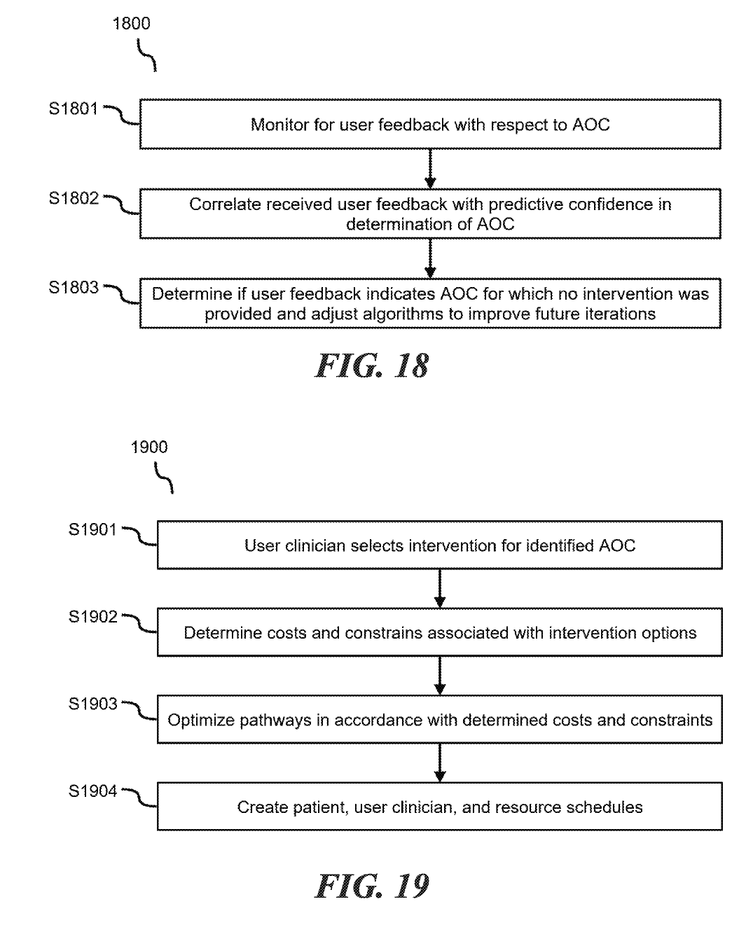

FIG. 18 is an example of a methodology for adjusting the prioritization of Areas of Concern based upon subjective feedback from one or more user clinicians in accordance with one aspect of the present invention. The method 1800 begins at S1801 when the system monitors for clinician feedback with respect to the prioritized Areas of Concern. In an embodiment, the system may provide the clinician options for voting up and/or down the confidence in a prioritized Area of Concern. In a further embodiment, the system may provide the clinician options for selecting non-prioritized Areas of Concern to associate with a given patient. The system may aggregate clinician feedback from multiple users, such as, for example, a patient's care team.

In step S1802, the system correlates received user feedback with predictive confidence in the identified Area of Concern and the algorithms used to identify said Areas of Concern. In an embodiment, the system may determine a confidence interval in association with the positive and negative feedback provided by one or more users and adjust the prioritization of Areas of Concern in accordance with the determined confidence. For example, where multiple users indicate positive feedback for accurate prediction of a prioritized Area of Concern, the system may increase a confidence variable associated with the predictive algorithm prioritizing that Area of Concern and may further prioritize it in future iterations over comparatively less confident prioritizations of other Areas of Concern. In one embodiment, the system may determine a 95% confidence interval for the calculation of predictive confidence. In another embodiment, the system may adjust the predictive confidence impact in accordance with the time and/or point on an Area of Concern pathway. For example, the system may programmatically weigh early negative feedback from a user clinician higher than late negative feedback from a user clinician to reflect the higher degree of confidence in rule-out factors.

In step S1803, the system determines if the feedback provided by the clinician user indicates the prioritization and/or presence of an Area of Concern for which no intervention was provided and adjust the algorithms and/or algorithmic variables of the Area of Concern prioritization and/or association of interventions with Areas of Concern to improve accuracy in further iterations of the method. For example, the system may de-prioritize Areas of Concern for which multiple clinician users provided negative feedback. In an embodiment, the system may determine whether the timing of the feedback indicates clinician rule-out of a specific Area of Concern and adjusts the predictive Area of Concern algorithms accordingly.

FIG. 19 is an example of a methodology for identifying treatment pathways and creating schedules for a selected intervention for one or more Areas of Concern. Method 1900 begins at a first step S1901 wherein the clinician user selects an intervention for an identified Area of Concern. Alternatively, the system may automatically select one or more interventions associated with an Area of Concern, such as, for example, selection of interventions with a likelihood of performance above a certain threshold value.

In step S1902, the system determines the costs and constraints associated with various intervention options for each decision step associated with the intervention. For example, an intervention may contemplate the performance of an imaging test for the patient for evaluation of a treatment plan, wherein a decision factor is the conducting of a CT scan, an MRI, or a PET scan. For this decision factor and each potential conduit, the system may determine the cost of each scan and constraints associated with each scan, such as, for example, availability of scheduling of each machine and availability of clinician operators. In an embodiment, the system may determine constraints with respect to patient attributes, such as, for example, determining for a pregnant patient a higher risk factor for the PET scan on the basis of the use of a radiotracer and a higher risk factor for the CT scan on the basis of the exposure to x-rays when compared to the MRI scan.

In step S1903, the system optimizes pathways in accordance with the determined costs and constraints. In an embodiment, the system may present to the user clinician one or more suggested pathways as a compilation of suggested pathway decision points for the user to verify. The user may further be able to adjust the pathway and suggested decision points, e.g., selecting an MRI scan or a specific medical expert to conduct a clinical review. In an embodiment, the user may be presented a pathway in the form of a branching decision tree.

In step S1904, the system creates one or more patient schedules, user clinician schedules, and resource schedules in accordance with the determined pathway. In one embodiment, the system may further update relevant medical system databases with date and calendar information pursuant to the schedules and determined pathway, such as, for example, booking an appointment with an outpatient facility and medical specialist.

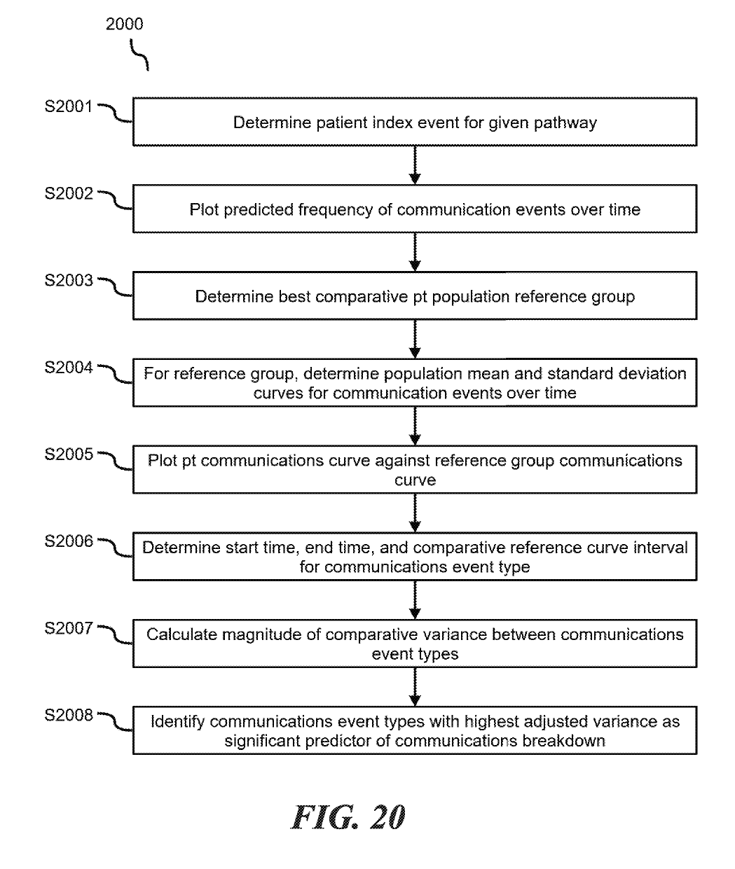

FIG. 20 is an example of a methodology for determining, for a given treatment pathway, communication events associated with a high risk of causing a communication breakdown likely to cause medical error. Method 2000 begins at a first step S2001 wherein the system determines an index event for a given pathway and patient. Index events may be, for example, hospitalization, new diagnosis, start of therapy or procedure, and the like. Communications events may be one or more categories, sets, or subsets of messages, e.g. messages containing a specific word or terminology in its content, messages categorized as a certain priority, and/or messages between one or more users or groups of users. In an embodiment, communications events may be non-message user interactions with the system, e.g. views of a patient digest, generation of patient clinical summary, generation of patient PSA, and the like.

In step S2002, the system plots a predicted frequency of communications events over time starting from the patient index event. In an embodiment, the system may visually plot the frequency of communications events over time via a graphical user interface for clinician review.

In step S2003, the system determines the best comparative patient population reference group for the selected patient for the plotting of frequency of communications events over time. For example, comparative patient population groups may be patients with the same first listed diagnosis, patients undergoing the same procedure, patients prescribed the same medications, patients with the same attending doctor, etc. In an embodiment, the determination of best reference group may be in accordance with logistic regression models wherein the population reference group with the greatest concordance statistic (i.e. C-index, C-statistic) is determined as the best comparative match. In various embodiments, machine learning algorithms may be used to optimize identification and/or matching of comparative patient population reference groups. For example, machine learning algorithms may identify a prior-undefined group of patients where such patients have a comorbidity score of 5 admitted by Cardiology.

In step S2004, for the determined best reference group, the system determines population mean and standard deviation curves for communications events over time. In one embodiment, the system may determine the curves from historical analysis of messaging data and/or communications events data for the reference group. In another embodiment, the system may predict one or more of the mean and/or standard deviation curves via subsidiary iterations of method 2000.

In step S2005, the system plots the population mean and standard deviation curves for communications events over time against the patient communications curve. For example, the system may plot the reference group's communications curves for 0 (mean), −1σ, −2σ, −3σ, +1σ, +2σ, and +3σ. In an embodiment, the system may visually plot the curves for user review via a graphical user interface.

In step S2006, the system determines a start time, end time, and comparative reference curve interval (i.e. +/−σ) for a given communication event type. In an embodiment, the start time, end time, and comparative reference curve interval may be determined in accordance with logistic regression models wherein the times and curve intervals with the greatest concordance statistic is determined as the best comparative match. In various embodiments, machine learning algorithms may be used to optimize identification and/or matching of the start time, end time, and comparative reference curve interval. For example, machine learning algorithms may identify a start time of tx, an end time of ty, and a comparative reference curve interval of +/−σ1 for unhandled STAT messages, whereas messages containing the word “discharge” may instead have a start time of ty, an end time of tz, and a comparative reference curve interval of +/−σ2.

In step S2007, the system calculates a magnitude of comparative variance between the predicted patient communication curve and the comparative reference curve interval bounded by the start and end time for each communication event type. In various embodiments, one or more variable weights may be applied to the area calculations for determining significance of communication breakdown events. For example, communications events with a known high correlation to communications breakdown and/or medical error may result in a higher multiplier variable weighing the bounded area between the predicted patient communication curve and comparative reference curve. In an embodiment, the determination of communications events to include in analysis and the weight to apply to the area calculated for each communication event may be determined in accordance with logistic regression models.

In step S2008, the system identifies the communications event types with the highest adjusted variance and determines these communications event types as predictors of communications breakdown. Where a patient's predicted communication curve deviates significantly from the historical norm or predicted comparative norm, such deviation may be a strong predictor of communications breakdown that may lead to medical error. By applying the boundaries of the start time, end, time, and comparative reference curve interval, as well as any variable weights to scale for magnitude or criticality of breakdowns associated with the communications events, the system predicts where critical deviations may occur in advance. Accordingly, prediction of communications breakdown in this manner may be displayed to a clinician user via the user interface and may further adjust routing of messages to an optimal recipient on the basis of the predicted breakdown. For example, the system may generate an alert for one or more users warning of potential communications breakdown and suggesting remedial action (e.g. modifying a selected intervention pathway), or the system may change message routing preferences in a manner that minimizes the likelihood of communications breakdown (e.g. alerting a nurse clinician as opposed to a doctor clinician where the doctor clinician is unlikely or unable to take requisite action).

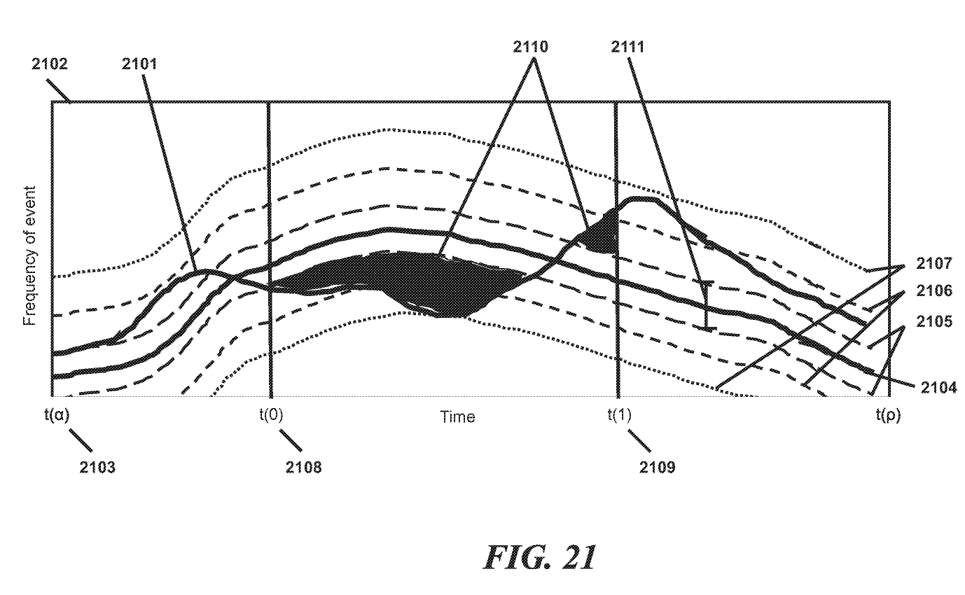

FIG. 21 is a modified screen shot representing an exemplary comparative communications event plot according to the present invention and may be interpreted in accordance with method 2000 above. A patient communications curve 2101 is plotted in accordance with graph of communications event frequency over time 2102 beginning from a patient index event 2103. A curve of the mean communications events for a patient reference group 2104 is comparatively plotted, as are one or more curves of the standard deviation communication events for the same patient reference group (2105, 2106, 2107). For a communications event type, a start time 2108 and end time 2109 are identified as left-most and right-most bounds of an area plot 2110, and a reference curve interval 2111 determines the upper and lower bounds of the area plot. The area plot 2110 delineates the difference between the patient communications curve 2101 and the determined reference curve interval 2111 indicating the time and deviation for which a series of communication events for a patient is expected to deviate from the norm and introduce a communications breakdown and potential medical error.

The previous detailed description has been provided for the purposes of illustration and description. Thus, although there have been described particular embodiments of systems and methods according to the present invention, it is not intended that such references be construed as limitations upon the scope of this invention except as set forth in the following claims.

What is claimed is:

1. A computer implemented method for patient handoff from a first healthcare provider associated with a first patient data interface, the method comprising:
enabling initiation of patient handoff from the first healthcare provider to a second healthcare provider;
linking a user computing device associated with the second healthcare provider to a server-linked database comprising a patient data record assembled thereon;
generating a second patient data interface on a display of the user computing device associated with the second healthcare provider, based at least on processing one or more attributes for the second healthcare provider and one or more patient attributes from the patient data record;
enabling, via user selection tools associated with the second patient data interface, healthcare provider feedback regarding the selection and arrangement of factors in the second patient data interface; and
determining whether to apply the received feedback at least in part for selection and arrangement of factors in subsequent patient handoff iterations.

2. The method of claim 1, wherein the second patient data interface is generated comprising a plurality of factors that are visually arranged therein.

3. The method of claim 2, wherein the second patient data interface is based on linear regression scores further accounting for attributes of the factors, wherein the attributes of the factors comprise an abnormal state or a normal state.

4. The method of claim 2, wherein the plurality of factors are selected for presentation from among a census of factors based on exceeding a threshold value.

5. The method of claim 4, wherein the threshold value is dynamic based on one or more of: a type of the second healthcare provider; received feedback from the second healthcare provider; and one or more patient specific outcomes.

6. The method of claim 1, further comprising generating a list of potential areas of concern in association with generation of the second patient data interface, the potential areas of concern ordered according to an area plot comprising inputs for provider feedback, patient-matched areas of concern, and predicted quality-adjusted life-year (QALY) impact.

7. The method of claim 6, wherein the provider feedback comprises positive or negative inputs for respective potential areas of concern listed in the second patient data interface.

8. The method of claim 6, wherein the QALY impact is theoretically derived based on predicted impacts of one or more interventions on length of life and quality of life for the patient.

9. The method of claim 1, further comprising selecting an intervention pathway based on the listed potential areas of concern and representing the intervention pathway in association with the generated second patient data interface.

10. The method of claim 9, further comprising prioritizing elements of the intervention pathway based on their respective predicted QALY impacts.

11. The method of claim 9, further comprising prioritizing elements of the intervention pathway based on selected patient-specific outcomes.

12. The method of claim 9, further comprising generating and presenting a resource schedule for implementation of one or more elements of the intervention pathway.

13. The method of claim 1, further comprising determining whether the received feedback requires modification to an algorithm for selection and presentation of factors in subsequent patient handoff iterations, and accordingly modifying the algorithm.

14. The method of claim 1, further comprising determining whether the received feedback requires modification of one or more data sources associated with selection and presentation of factors in subsequent patient handoff iterations, and accordingly generating an alert to at least the second healthcare provider.

15. The method of claim 1, further comprising:
mapping one or more patient data elements to respective human body parts in the server-linked database; and
generating as part of the second patient data interface a two-dimensional representation of the human body having the human body parts and the respective mapped patient data represented therewith.

16. The method of claim 15, further comprising:
sorting the mapped patient data for each of the respective human body parts in accordance with dates for corresponding data points; and
generating a dynamic chronological representation for each of the data points corresponding to the mapped patient data.
